(19) United States
(12) Patent Application Publication
(10) Pub. No.: US 2017/0274095 A1
(43) Pub. Date: Sep. 28, 2017

(54) ENHANCED REGULATORY T CELLS TARGETED TO SITES OF INFLAMMATION WITH CHIMERIC ANTIGEN RECEPTORS AND EXPRESSING FACTORS THAT ENHANCE VIABILITY OF PANCREATIC CELLS

(71) Applicant: The Board of Trustees of the Leland Stanford Junior University, Stanford, CA (US)

(72) Inventors: Everett Hurteau Meyer, Reston, VA (US); Panagiota Iliopoulou, San Carlos, CA (US); Antonio Pierini, Castiglione del Lago (PG) (IT); Seung K. Kim, Stanford, CA (US)

(21) Appl. No.: 15/466,591

(22) Filed: Mar. 22, 2017

Related U.S. Application Data
(60) Provisional application No. 62/312,331, filed on Mar. 23, 2016.

(51) Int. Cl. A61K 49/00; C07K 16/44; A61K 35/17; C07K 14/705; A61K 47/48; C07K 19/00

(52) U.S. Cl. CPC A61K 47/48723; A61K 35/17; C07K 16/44; C07K 14/70521; C07K 14/70503; C07K 2317/622; C07K 2319/00

(57) ABSTRACT

Regulatory T cells (Treg) are engineered to express a chimeric antigen receptor (CAR), that specifically binds to a non-endogenous antigenic moiety; and are administered in combination with an effective dose of targeting antibodies, which antibodies (i) bind to an antigen localized at a site of inflammation and (ii) are labeled with the antigenic moiety, wherein inflammation is decreased at the targeted site.

### CROSS REFERENCE

This application claims benefit of U.S. Provisional Patent Application No. 62/312,331, filed Mar. 23, 2016, which application is incorporated herein by reference in its entirety.

### BACKGROUND OF THE INVENTION

The concept of introducing into a cytotoxic T-cell hybridoma the genetic material for an antibody recognizing a model antigen (a hapten, 2,4,6-trinitrophenyl) was first described in 1989. The principles have since been applied to a number of tumor antigen specificities. At its simplest embodiment, a chimeric T cell antigen receptor (CAR) is a polypeptide comprising sequences of a light and heavy chain from an antibody, linked to the signaling machinery of the T-cell receptor, typically the ζ chain. Modifications of this design have led to the addition of costimulatory domains that are derived from one or more of the endogenous molecules used by T cells, such as CD27, CD28, CD134, or CD137. CAR constructs typically consists of an extracellular target-binding domain, a hinge region, a trans-membrane domain that anchors the CAR to the cell membrane, and one or more intracellular signaling domains. The target-binding domain is usually derived from the light and heavy chain portions of a single chain variable fragment (scFv). Affinity and avidity are much higher for CAR binding versus binding of a T cell receptor to its cognate antigen. CARs recognize cell surface proteins, and therefore targeting is not MHC-restricted. Furthermore, unlike TCR-based recognition, CAR recognition is not dependent on processing and antigen presentation.

The incorporation of costimulatory molecules such as CD27, CD28, CD134 (OX40), CD137 (4-1BB), CD244, or ICOS into a CAR can augment the effects of ζ chain signaling and enhance T-cell proliferation and persistence. Incorporation of a single costimulatory molecule has been found to lead to superior persistence and other T-cell functions.

The CAR construct can be introduced into T cells using viral or non-viral techniques. Gammaretroviral or lentiviral vectors integrate into the host cell genome and have low intrinsic immunogenicity and hence lead to permanent transgene expression. Other viral vectors include adenovirus or adeno-associated virus, which provide long-term episomal transgene expression and have been shown to infect human T cells with high efficiency, but have a disadvantage of immunogenicity. Non-viral approaches include transposon/transposase systems, such as Sleeping Beauty, that can deliver a large payload with persistent high-level transgene expression. Alternatively a DNA plasmid encoding the CAR can be transcribed in vitro, and the resulting mRNA electroporated into T cells.

T-cell trafficking is dependent upon an array of soluble factors, receptors, and adhesion molecules. Recruitment of effector T cells into the targeted microenvironment may be impeded by sub-threshold expression of homing and trafficking molecules on tumor microvessels and conversely may be enhanced by cytokine signaling. T cells can be transduced or electroporated to overexpress relevant chemokine and homing molecules to promote homing to desired tissues. See, for example Di Stasi, et al. Blood 2009; 113:6392-6402; Craddock et al. J Immunother 2010; 33:780-788. Strategies to enhance T-cell persistence after transfer include exogenous cytokine administration, overexpression of pro-survival signals, or the reversal of anti-survival signals.

Further methods of treating disease with engineered T cells is of great clinical interest. The present invention addresses this issue.

### SUMMARY OF THE INVENTION

Compositions and methods are provided for treating inflammatory disease, including autoimmune inflammatory disease, particularly diseases involving pancreatic tissue. Diabetic conditions are of particular interest, including, for example, insulin dependent diabetes mellitus (IDDM) and Type 2 diabetes. Other inflammatory conditions involving pancreatic tissue are also of interest, e.g. chronic pancreatitis, etc.

In the methods of the invention, regulatory T cells (Treg), are engineered to express a chimeric antigen receptor (CAR), that specifically binds to a non-endogenous antigenic moiety. In some embodiments, the antigenic moiety is a small molecule, e.g. a hapten, including without limitation fluorescein isothiocyanate (FITC), streptavidin, biotin, dinitrophenol, phycoerythrin (PE), and the like. The engineered T cell is further modified to express therapeutic levels of a factor that enhances pancreatic cell viability, function, etc. In some embodiments the factor is selected from GLP1, STIM1, prolactin, placental lactogen, PDGF A, PDGF B, GIP, and VEGFA. In some embodiments the factor is GLP1.

The engineered Treg cells are administered to an individual suffering from an inflammatory condition of the pancreas in combination with administration of targeting antibodies, which antibodies (i) bind to an antigen localized at the site of inflammation and (ii) are labeled with the antigenic moiety. By localizing Treg cells at the site of inflammation, the effectiveness of the Treg cells is enhanced. Targeting antibodies of interest include, without limitation, antibodies that specifically bind to antigens present on human endothelial cells, e.g. CD31, and antibodies that specifically bind to pancreatic antigens, e.g. islet cell epitopes including HIC-2B4, CD26, and the like.

In some embodiments of the invention, the Treg cells are natural Treg cells. The cells may be autologous or allogeneic with respect to the recipient. In some embodiments, Treg cells are isolated from a peripheral blood sample by selection for cells that express CD4 and CD25. In some embodiments, the cells are expanded in culture following introduction of the CAR genetic construct. Other immunoregulatory populations can be utilized such as induced Tregulatory cells Lag3+, PD-1+ invariant natural killer cells or TIM-1+ regulatory B cells.

In some embodiments the CAR construct encodes one or more costimulatory molecule(s). In some embodiments the costimulatory molecule comprises the CD28 activating domain. In some embodiments the CAR construct encodes or more T cell downregulatory proteins, such as an immune checkpoint protein. In some embodiments the immune checkpoint protein is CTLA4. In some embodiments the immune checkpoint protein is LAG3. In some embodiments both CTLA4 and LAG3 are expressed by the engineered Treg cell. In some embodiments, IL-2 pathway proteins are expressed by the engineered Treg cells.

In a related embodiment, a method of treating or preventing pancreatic disease, e.g. IDDM, the method comprising administering to a subject (i) an effective dose of Treg cells engineered to express a CAR that is specific for a non-endogenous antigenic moiety and (ii) an effective dose of a targeting antibody labeled with the non-endogenous antigenic moiety, wherein the antibody binds to an antigen present on pancreatic islet cells or endothelial cells; wherein the Treg cells localize in the pancreas and downregulate inflammatory T cell activity. In some embodiments, an effective dose of the engineered Treg cells and targeting antibodies is administered to a diabetic patient, or a pre-diabetic patient.

It has been surprisingly found that engineered Treg localization and allograft tolerization persisted long after transient expression of the CAR construct. For example, a one-time introduction of transiently expressing CAR Treg resulted in antigen-specific peripheral tolerance for an extended period of time. mAbCAR-mediated transient binding induces antigen specificity that persists even if the initiating mAbCAR function itself is lost. In some embodiments, expression of the mAbCAR construct by an engineered T cell is transient, e.g. detectable expression is present for less than about 4 weeks, less than about 3 weeks, less than about 2 weeks, less than about 1 week. In some embodiments antigen-specific peripheral tolerance is provided for greater than about 2 weeks, greater than about 3 weeks, greater than about 1 month, greater than about 2 months, greater than about 3 months, greater than about 4 months, greater than about 6 months, or more.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1D. mAbCAR construct and its expression in transfected T cells and Treg. FIG. 1A Schematic representation of mAbCAR construct. FIG. 1B Model of mAbCAR molecule expression by transfected cells. FIG. 1C mAbCAR expression by untransfected or transfected T cells and Treg measured by anti-FLAG and different FITC-conjugated mAbs as indicated. FIG. 1D Kinetic of mAbCAR expression over time in T cells after transient transfection.

FIG. 2A-2C. mAbCAR expressing T cells are activated by FITC binding. FIG. 2A(a)-(b) Mass cytometry analysis of mAbCAR expressing CD4⁺ and CD8⁺ T cells. Bars represent percentage of expression of selected markers (FoxP3, CD25, CD44, CD62L, CD69, CD103, CD127, IFNγ, LAG3, PD1, TIM1) after in vitro exposure to FITC. Data show marker expression in untransfected CD4⁺ FIG. 2A(a) and CD8⁺ FIG. 2A(b) T cells (white bars), isotype-mAbCARCD4⁺ and CD8⁺ T cells (grey bars), MAdCAM1-mAbCAR-CD4⁺ and CD8⁺ T cells (black bars) FIG. 2B CD25 and CD69 surface expression is also reported in mAbCAR T cells loaded with FITCisotype control ab or FITC-anti-MAdCAM1 mAb and cultured with irradiated cell suspension derived from syngeneic spleen where MAdCAM1 was also expressed. FIG. 2C(a)-(b) Percentage FIG. 2C(a) and FACS plot FIG. 2C(b) of naïve (CD62L₊CD44₋), central memory (CD62L₊CD44₊), and effector memory (CD62L₋CD44₊) in vitro cultured FITC-isotype or FITCMAdCAM1-mAbCAR T cells have been also reported.

FIG. 3A-3D. Tissue specific FITC-mAbs modulate mAbCAR T cells homing and function in vivo. FIG. 3A(a)-(b) Bars FIG. 3A(a) represent BLI signal from luc+ untransfected T cells (black), luc₊ SDF1-mAbCAR T cells (white), or luc₊ MAdCAM1-mAbCAR T cells (grey) at day +4, +7 and +12 after adoptive transfer in lethally irradiated allogeneic mice that received TCD BM at day 0. Representative BLI images FIG. 3A(b) at day +12 after transfer are also reported. FIG. 3B GvHD score over time of recipient mice that received allogeneic untransfected T cells, SDF1-mAbCAR T cells, or MAdCAM1-mAbCAR T cells. FIG. 3C(a)-(b) Tumor growth analyzed by BLI uptake FIG. 3C(a) in lethally irradiated BALB/c mice that received allogeneic C57BL/6 TCD BM and luc₊ A20 alone (striped bars), luc₊ A20 and allogeneic isotype-mAbCAR T cells (white bars), or luc₊ A20 and allogeneic SDF1-mAbCAR T cells (black bars). Representative BLI images FIG. 3C(b) at day +7, +14, and +21 are also reported. FIG. 3D Survival of lethally irradiated BALB/c that received allogeneic C57BL/6 TCD BM and luc₊ A20 alone, luc₊ A20 and allogeneic isotype-mAbCAR T cells, or luc₊ A20 and allogeneic SDF1-mAbCAR T cells. Survival of control mice that received irradiation alone and mice that received irradiation and allogeneic TCD BM is also reported.

FIG. 4A-4G. mAbCAR-Treg retain phenotype and function. FIG. 4A FoxP3 intranuclear expression measured through mean fluorescence intensity (MFI) in untransfected Treg (white bar), transfected Treg incubated with FITC-isotype control antibody (grey bar), and transfected Treg incubated with FITC-MAdCAM1 antibody (black bar). Histogram overlap of FoxP3 expression is also reported. FIG. 4B Mass cytometry analysis of mAbCAR expressing CD4₊FoxP3₊ Treg. Bars represent percentage of expression of selected markers relevant for Treg function (FoxP3, CD25, CD44, CD62L, CD69, CD103, CD127, CTLA4, GATA3, Helios, LAG3, PD1, TBET, TIM1) before or after in vitro exposure to FITC. Data show marker expression in untransfected Treg (white bars), FITC-isotype-mAbCAR Treg (grey bars), FITC-MAdCAM1-mAbCAR Treg (black bars). FIG. 4C Frequency of phosphorylated STAT5 expression in untransfected (white bars) and transfected (black bars) Treg. FIG. 4D TCRβ repertoire of Treg analyzed by comparing TCRβ clone frequency of untransfected (y axis) and transfected (x axis) Treg. FIG. 4E Percentage of cell profileration of untransfected Treg (white bar), FITC-isotype-mAbCAR Treg (grey bar), and FITC-MAdCAM1-mAbCAR Treg (black bar) after culture with anti-CD3/CD28 beads measured through cell trace violet dilution. Sample FACS analysis is also shown. FIG. 4F Percentage of suppression of T cell proliferation stimulated with irradiated allogeneic splenocytes measured through cell trace violet analysis by untransfected Treg, FITC-isotype-mAbCAR Treg, FITC-MAdCAM1-mAbCAR Treg at T cell:Treg ratio of 1:1, 1:2, 1:4, and 1:8. FIG. 4G Survival of lethally irradiated BALB/c mice that received allogeneic TCD BM alone, TCD BM+Tcon+untransfected Treg, TCD BM+Tcon+mAbCAR Treg, and TCD BM+Tcon+FITC-isotype-mAbCAR Treg. Survival of control mice that received irradiation alone is also reported.

FIG. 5A-5D. FITC-H-2D^{d}-mAbCAR Treg induce tolerance to allogeneic pancreatic islet grafts if directed against the islet MHC-I alloantigen. FIG. 5A experimental scheme FIG. 5B(a) Fold change over time of BLI uptake (radiance) from mice that received luc₊ pancreatic islet graft alone, luc₊ pancreatic islet graft+FITC-isotype-mAbCAR Treg, and luc₊ pancreatic islet graft+FITC-H-2D_{d}-mAbCAR Treg is reported. FIG. 5B(b) Representative images at 4 weeks after islet transplant are also reported FIG. 5C Percentage of pancreatic islet graft survival at 6 weeks after transplant in mice that received no Treg treatment (white bar), FITC-isotype-mAbCAR Treg (grey bar), FITC-H-2D_{d}mAbCAR Treg (black bar). FIG. 5D Percentage of pancreatic islet graft infiltration by host type CD8₊ T cells in mice that received no Treg treatment (white bar), FITC-isotype-mAbCAR Treg (grey bar), FITC-H-2D_{d}mAbCAR Treg (black bar) at 10 days after transplant.

FIG. 6A-6D. FITC-H-2D^{d}-mAbCAR Treg home and expand in proximity to allogeneic pancreatic islet grafts. FIG. 6A(a)-(b) Bars represent BLI signal from standardized regions of interest in left kidney area in sublethally irradiated mice that received a graft of allogeneic pancreatic islets alone (white bars), pancreatic islets+luc₊ FITC-isotype-mAbCAR Treg (grey bars), and pancreatic islets+luc₊ FITC-H-2D_{d}-mAbCAR Treg (black bars), at day +3, +5, +7 and +10 after adoptive transfer. Representative BLI images are also reported. FIG. 6B Representative histologic sections of allogeneic pancreatic grafts in mice that received no Treg treatment (left panels), GFP₊-FITC-isotype-mAbCAR Treg (middle panels), or GFP₊-FITC-H-2D_{d}-mAbCAR Treg (right panels) 10 days after transplantation and Treg transfer. Hematoxilin-Eosin staining (upper panels) shows well preserved grafts in mice that received Treg treatments (arrows=pancreatic islets in kidney capsule). Confocal microscopy analysis (lower panels) demonstrates presence of transferred Treg (GFP⁺, green, arrows) in proximity of islet grafts (insulin, red) only in mice that received GFP⁺-FITC-H-2D^{d}-mAbCAR Treg. FIG. 6C Sample FACS analysis showing percentage of transferred GFP⁺ Treg in spleens of mice that received no Treg treatment, FITC-isotype-mAbCAR Treg, and FITC-H-2D^{d}-mAbCAR Treg at 10 days after transplant. FIG. 6D MFI of CD25 and CD69 in previously transferred GFP⁺ Treg reisolated from spleens of mice that received FITC-isotype-mAbCAR Treg (grey bar) or FITC-H-2D^{d}-mAbCAR Treg (black bar) at 10 days after transplant.

FIG. 7A-7B. FITC-H-2D^{d}-mAbCAR-Treg acquire antigen specificity after in vivo transfer. FIG. 7A Representative pictures over time of mice that received a secondary double skin graft MHC-matched with the previously transplanted pancreatic graft (upper grafts) or “third-party” (lower grafts). FIG. 7B skin graft survival in mice that received pancreatic islet graft and no Treg treatment (skin MHC-matched with islet graft, skin “third-party”, mice that received pancreatic islet graft and FITC-isotype-mAbCAR Treg (skin MHC-matched with islet graft, skin “third party”, and mice that received pancreatic islet graft and FITC-H-2D^{d}-mAbCAR Treg (skin MHC-matched with islet graft, skin “third-party”. 1 of 3 consecutive experiments has been reported. 1 of the 3 experiments has been performed by transplanting skins in inverted position (“third-party” skins as upper grafts and MHC-matched skins as lower graft) in order to avoid technical bias.

FIG. 8. Mass cytometry reveals FITC induced activation of mAbCAR T cell subsets Spade analysis of mAbCAR T cells that have been analyzed through mass cytometry after incubation with FITC conjugated isotype or anti-MAdCAM1 antibodies revealed increased CD25, CD69 and LAG3 expression in central memory (CD44₊CD62L₊, blue arrows) and effector memory (CD44₊CD62L_{neg}, red arrows) subsets of CD4₊FoxP3_{neg} T cells and CD4₊FoxP3₊ Treg cells. Reported analysis has been performed on the live CD4₊ T cell population and cells have been gated for CD4₊FoxP3₋ cells, CD4₊FoxP3₊ cells as indicated. Shown data is one representative sample of three samples of CD4₊ T cells cultured in the presence or not of FITC conjugated isotype or anti-MAdCAM1 antibodies. Dot size is representative of the size of the single homogenous cell population. Plot color intensity refers to the grade of the expression of the reported markers as shown. Data are representative of one of two consecutive experiments.

FIG. 9A-9D. mAbCAR T cells do not show different patterns of homing marker expression after in vivo transfer Surface expression of CXCR4 FIG. 9A, LAMP1 FIG. 9B, CD62L FIG. 9C, CXCR5 FIG. 9D in CD4₊ (white bars) and CD8₊ T cells (black bars) reisolated from transplanted mice that received adoptive transfer of untransfected T cells, mAbCAR T cells, FITCMAdCAM1 mAbCAR T cells, or FITC-SDF1 mAbCAR T cells is reported. Data are representative of one of two consecutive experiments.

FIG. 10. Ex vivo BLI demonstrates higher frequencies of H-2D_{d}-mAbCAR Treg cells in kidneys transplanted with allogeneic pancreatic islet grafts BLI signal is reported from left kidney of mice that received luciferase₊ H-2D_{d}mAbCAR Treg cells (black bar) or isotype-mAbCAR Treg cells (white bar) and allogeneic pancreatic islet graft in the right kidney capsule. A representative image is also reported were the transplanted kidneys are shown in the right side of the pictures and control kidneys in the left side.

FIG. 11. H-2D_{d}-mAbCAR Treg cells reduce occurrence of rejection in H-2D_{d+} skin grafts Hystologic sections of different skin grafts as reported at day +14 after skin transplant. Hematoxilin-Eosin staining is shown. Skin MHC-matched with pancreatic islet graft from mice that were treated with FITC-H-2D_{d}-mAbCAR-Treg show less signs of graft rejection (lymphoid infiltration, full arrows; picnotic bodies, bold arrows) and better persistence of normal subcutaneous adipose tissue (*).

### DETAILED DESCRIPTION OF THE EMBODIMENTS

The present invention provides engineered T cells that express a chimeric antigen receptor (mabCAR) that binds to and is activated by targeting monoclonal antibodies, including without limitation binding to a small molecule tag present on the targeting antibodies. The T cells are targeted to pancreatic cells, and are further modified to express factors that enhance viability and/or function of pancreatic cells.

Highly selective targeted T cell therapies are effective non-toxic modalities for the treatment of various conditions. Inflammatory conditions, such as IDDM, are complex diseases where multiple elements contribute to the overall pathogenesis through both distinct and redundant mechanisms. Hence, targeting different markers alone or in combination could result in better therapeutic efficacy. However, developing separate cellular products for clinical use can be impractical, owing to regulatory hurdles and cost. In contrast, rendering an individual T cell to be specific for a tag that can be conjugated to different antibodies provides flexibility and reduced cost.

### Definitions

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

General methods in molecular and cellular biochemistry can be found in such standard textbooks as Molecular Cloning: A Laboratory Manual, 3rd Ed. (Sambrook et al., CSH Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

By “comprising” it is meant that the recited elements are required in the composition/method/kit, but other elements may be included to form the composition/method/kit etc. within the scope of the claim.

By “consisting essentially of”, it is meant a limitation of the scope of composition or method described to the specified materials or steps that do not materially affect the basic and novel characteristic(s) of the subject invention.

By “consisting of”, it is meant the exclusion from the composition, method, or kit of any element, step, or ingredient not specified in the claim.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered during the symptomatic stage of the disease, and in some cases after the symptomatic stage of the disease.

A “therapeutically effective amount” is intended for an amount of active agent which is necessary to impart therapeutic benefit to a subject. For example, a “therapeutically effective amount” is an amount which induces, ameliorates or otherwise causes an improvement in the pathological symptoms, disease progression or physiological conditions associated with a disease or which improves resistance to a disorder.

The term “genetic modification” means any process that adds, deletes, alters, or disrupts an endogenous nucleotide sequence and includes, but is not limited to viral mediated gene transfer, liposome mediated transfer, transformation, transfection and transduction, e.g., viral mediated gene transfer such as the use of vectors based on DNA viruses such as lentivirus, adenovirus, retroviruses, adeno-associated virus and herpes virus.

“Variant” refers to polypeptides having amino acid sequences that differ to some extent from a native sequence polypeptide. Ordinarily, amino acid sequence variants will possess at least about 80% sequence identity, more preferably, at least about 90% homologous by sequence. The amino acid sequence variants may possess substitutions, deletions, and/or insertions at certain positions within the reference amino acid sequence.

“Antibody-dependent cell-mediated cytotoxicity” and “ADCC” refer to a cell-mediated reaction in which nonspecific cytotoxic cells that express Fc receptors, such as natural killer cells, neutrophils, and macrophages, recognize bound antibody on a target cell and cause lysis of the target cell. ADCC activity may be assessed using methods, such as those described in U.S. Pat. No. 5,821,337.

“Effector cells” are leukocytes which express one or more constant region receptors and perform effector functions.

As used herein, the term “subject” denotes a mammal, such as canines; felines; equines; bovines; ovines; etc. and primates, particularly humans. Animal models, particularly small mammals, e.g. murine, lagomorpha, etc. can be used for experimental investigations. Preferably a subject according to the invention is a human.

A “cytokine” is a protein released by one cell to act on another cell as an intercellular mediator.

“Non-immunogenic” refers to a material that does not initiate, provoke or enhance an immune response where the immune response includes the adaptive and/or innate immune responses.

The term “gene” means the segment of DNA involved in producing a polypeptide chain; it includes regions preceding and following the coding region “leader and trailer” as well as intervening sequences (introns) between individual coding segments (exons). Some genes may be developed which lack, in whole or in part, introns. Some leader sequences may enhance translation of the nucleic acid into polypeptides.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

Inflammatory Disease.

Inflammation is a process whereby the immune system responds to infection or tissue damage. Inflammatory disease results from an activation of the immune system that causes illness, in the absence of infection or tissue damage, or at a response level that causes illness. Inflammatory disease includes autoimmune disease, which are any disease caused by immunity that becomes misdirected at healthy cells and/or tissues of the body. Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body.

The immune system employs a highly complex mechanism designed to generate responses to protect mammals against a variety of foreign pathogens while at the same time preventing responses against self-antigens. In addition to deciding whether to respond (antigen specificity), the immune system must also choose appropriate effector functions to deal with each pathogen (effector specificity). A cell critical in mediating and regulating these effector functions are CD4⁺ CD25⁺ T regulatory cells.

Human insulin-dependent diabetes mellitus (IDDM) is a disease characterized by autoimmune destruction of the β cells in the pancreatic islets of Langerhans. An animal model for the disease is the non-obese diabetic (NOD) mouse, which develops autoimmunity. NOD mice spontaneously develop inflammation of the islets and destruction of the β cells, which leads to hyperglycemia and overt diabetes. Both CD4+ and CD8+ T cells are believed to be required for diabetes to develop: CD4+ T cells appear to be required for initiation of insulitis, cytokine-mediated destruction of β cells, and probably for activation of CD8+ T cells. The CD8+ T cells in turn mediate β cell destruction by cytotoxic effects such as release of granzymes, perforin, TNF α and IFN γ.

The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic β cell function. The disease progression may be monitored in individuals diagnosed by family history and genetic analysis as being susceptible. The most important genetic effect is seen with genes of the major histocompatibility locus (IDDM1), although other loci, including the insulin gene region (IDDM2) also show linkage to the disease (see Davies et al, supra and Kennedy et al. (1995) Nature Genetics 9:293-298).

Markers that may be evaluated during the presymptomatic stage are the presence of insulitis in the pancreas, the level and frequency of islet cell antibodies, islet cell surface antibodies, aberrant expression of Class II MHC molecules on pancreatic β cells, glucose concentration in the blood, and the plasma concentration of insulin. An increase in the number of T lymphocytes in the pancreas, islet cell antibodies and blood glucose is indicative of the disease, as is a decrease in insulin concentration. After the onset of overt diabetes, patients with residual β cell function, evidenced by the plasma persistence of insulin C-peptide, may also benefit from the subject treatment, to prevent further loss of function.

The subject therapy will desirably be administered during the presymptomatic or preclinical stage of the disease, and in some cases during the symptomatic stage of the disease. Early treatment is preferable, in order to prevent the loss of function associated with inflammatory tissue damage. The presymptomatic, or preclinical stage will be defined as that period not later than when there is T cell involvement at the site of disease, e.g. islets of Langerhans, synovial tissue, thyroid gland, etc., but the loss of function is not yet severe enough to produce the clinical symptoms indicative of overt disease. T cell involvement may be evidenced by the presence of elevated numbers of T cells at the site of disease, the presence of T cells specific for autoantigens, the release of performs and granzymes at the site of disease, response to immunosuppressive therapy, etc.

Regulatory T Cells.

Regulatory T cells (“Treg”) are a specialized subpopulation of T cells which suppresses activation of the immune system and thereby maintains tolerance to self-antigens. There are various types of regulatory T cells. The majority of recent research has focused on TCRαβ+CD4+ regulatory T cells. These include natural regulatory T cells (nTreg), which are T cells produced in the thymus and delivered to the periphery as a long-lived lineage of self-antigen-specific lymphocytes; and induced regulatory T cells (iTreg), which are recruited from circulating lymphocytes and acquire regulatory properties under particular conditions of stimulation in the periphery. Both cell types are CD4+CD25+, both can inhibit proliferation of CD4+CD25− T cells in a dose dependent manner, and both are anergic and do not proliferate upon TCR stimulation. In addition to being positive for CD4 and CD25, regulatory T cells are positive for the transcription factor Foxp3, an intracellular marker.

In the methods of the invention, Treg cells can be isolated from a patient sample by selection for the phenotype of interest, e.g. for CD4+CD25+ cells. It will be understood by those of skill in the art that the stated expression levels reflect detectable amounts of the marker protein on the cell surface. A cell that is negative for staining (the level of binding of a marker specific reagent is not detectably different from an isotype matched control) may still express minor amounts of the marker. And while it is commonplace in the art to refer to cells as “positive” or “negative” for a particular marker, actual expression levels are a quantitative trait. The number of molecules on the cell surface can vary by several logs, yet still be characterized as “positive”. Any suitable method can be used, e.g. flow cytometry, panning, magnetic bead selection, and the like as known in the art. The cells can be expanded in culture before or after introduction of the CAR construct, e.g. by culture with anti-CD3 antibodies (for TCR stimulation) and excess exogenous IL-2 (a T cell growth factor). The anergic state of regulatory T cells can also be overcome by anti-CD28 costimulation or interaction with mature dendritic cells.

In some embodiments, methods of inducing the proliferation of regulatory T cells are employed to produce an enriched population of engineered Treg cells. By an “enriched population of regulatory T cells”, it is meant that the representation of regulatory T cells in the cell population is greater than would otherwise be, e.g., in the absence of the methods provided. In other words, methods of the invention increase the percentage of regulatory T cells in the population by at least 1.5 fold or more, e.g. 2-fold or more, in some instances 3-fold or more, relative to the number of regulatory T cells that would exist in the cell population in the absence of enrichment.

Antibody:

As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure.

The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)₂, Fab, Fv, rIgG, and scFv fragments. The term scFv refers to a single chain Fv antibody in which the variable domains of the heavy chain and of the light chain of a traditional two chain antibody have been joined to form one chain.

The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation.

Any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, antibody sequence elements are humanized, primatized, chimeric, etc, as is known in the art.

The use of a single chain variable fragment (scFv) is of particular interest for developing a CAR construct. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference.

“Receptor” means a polypeptide that is capable of specific binding to a molecule. Whereas many receptors may typically operate on the surface of a cell, some receptors may bind ligands when located inside the cell (and prior to transport to the surface) or may reside predominantly intra-cellularly and bind ligand therein.

Targeting antibody. A targeting antibody specifically binds to targeting antigen found in the region (or lesion) of the undesirable inflammation. It will be understood by one of skill in the art that a wide variety of antigens are available for this purpose, depending on the specific condition that is treated. In some embodiments the targeting antibodies specifically bind to an MHC Class I protein, e.g. HLA-A, HLA-B, HLA-C, etc.

Antigens associated with diabetes and pancreatic islets that may be used for targeting antibodies may include, without limitation, CD26, HIC-2B4, insulin, proinsulin, glutamic acid decarboxylase 65 (GAD65); islet cell antigen (ICA512; ICA12); ZnT8, IA-2; IA-2beta; HSP; glima 38; ICA69; and p52. In some embodiments the targeting antibodies specifically bind to a secreted factor, e.g. insulin, glucagon, SDF-1 or MCP-1.

In some embodiments the targeting antigen is an adhesion molecule involved in leukocyte trafficking, e.g. an integrin, a selectin, etc. Of interest are adhesion molecules expressed on endothelial cells, e.g. CD31, MADCAM-1, ICAM-1, VCAM-1, vascular adhesion protein 1 (VAP-1), fibronectin, paxillin, etc.

ICAM-1 (CD54) is a type I integral membrane glycoprotein with repeating Ig domains in the extracellular region, a structural signature of the Ig superfamily. Heterogeneity among different cell types gives rise to Mᵣ for ICAM-1 of 97 to 114 kd, most likely resulting from differential patterns of glycosylation. The non-N-glycosylated form has an Mᵣ of 55,000. A second ICAM isoform, ICAM-2 (CD102; Mᵣ 55-65 kd), is partially homologous to ICAM-1 but has only 2 Ig-like extracellular domains compared with 5 such domains for ICAM-1. ICAM-1 is believed important for leukocyte recruitment during a wide range of inflammatory and noninflammatory circumstances.

VCAM (CD106, Mᵣ 100-110 kd) is expressed by activated ECs and follicular dendritic cells. The VCAM extracellular domain contains repeating Ig domains, but because of alternate posttranscriptional splicing there are 2 VCAM messenger RNAs, a more abundant full length transcript and a variant that lacks exon 5. The VCAM variant maintains the same cytoplasmic domain but has a shorter extracellular domain.

The class of IgCAMS is of interest. These molecules can be involved in the adhesion of leukocytes, e.g. via LFA/ICAM-1, VLA-4/VCAM-1, etc. The molecules include the following:

| Molecule | Ligands | Distribution |
|---|---|---|
| ALCAM (CD166) | CD6; CD166; | Neural; Leukocytes |
| | NgCAM; 35 kD | |
| | protein | |
| Basigin (CD147) | | Leukocytes; RBCs; |
| | | Platelets; Endothelial |
| | | cells |
| BL-CAM (CD22) | Sialylated | B-Lymphocytes |
| | glycoproteins | |
| | LCA (CD45) | |
| CD44 | Hyaluronin; | Lymphocytes; Epithelial; |
| | Ankyrin; | WM perivascular astrocytes |
| | Fibronectin; | |
| | MIP1β | |
| | Osteopontin | |
| ICAM-1 (CD54) | αLβ2; | Leukocytes; Endothelial |
| | LFA-1 | cells; |
| | | Dendritic cells; Fibro- |
| | | blasts; Epithelium; |
| | | Synovial cells |
| ICAM-2 (CD102) | αLβ2 | Endothelial cells; |
| | (LFA-1) | Lymphocytes; Monocytes |
| ICAM-3 (CD50) | αLβ2 | Leukocytes |
| Lymphocyte function | LFA-3 | Lymphocytes; Thymocytes |
| antigen-2 (LFA-2) (CD2) | | |
| LFA-3 (CD 58) | LFA-2 | Leukocytes; Stroma |
| | | Endothelial cells |
| | | Astrocytoma |
| MAdCAM-1 | α4β7; | Mucosal endothelial |
| | L-selectin | cells |
| PECAM (CD31) | CD31; αvβ3 | Leukocytes; Synovial cells |
| | | Endothelial cells |
| VCAM-1 | α4β1; | Satellite cells |
| | α4β7 | Monocytes; Synovial cells; |
| | | Activated endothelial cells |

Also included are monoclonal antibodies having specificity for an integrin, e.g. a β2 integrin, a β1 integrin, a β7 integrin, an αI, αM, αX, αD, α4, α9, αv, etc. Also included are antibodies having specificity for pancreatic cells, pancreatic islet cells.

Non-Endogenous Antigenic Moiety.

A non-endogenous antigenic moiety if an antigen not normally present in the body. Typically the moiety is of a sufficiently small size that it can be used as a label, or tag, on a monoclonal antibody, but is of a size sufficient to bind to the mabCAR protein.

In some embodiments, the antigenic moiety is a small molecule, e.g. a hapten, including without limitation fluorescein isothiocyanate (FITC), streptavidin, biotin, dinitrophenol, phycoerythrin (PE), green fluorescent protein, horseradish peroxidase, histidine, streptavidin, fluorescent tags, and the like.

The antigenic moiety may be conjugated to the targeting antibodies using techniques such as chemical coupling and chemical cross-linkers. Alternatively, polynucleotide vectors can be prepared that encode the targeting antibodies as fusion proteins.

As used herein, a “vector” may be any agent capable of delivering or maintaining nucleic acid in a host cell, and includes viral vectors (e.g. retroviral vectors, lentiviral vectors, adenoviral vectors, or adeno-associated viral vectors), plasmids, naked nucleic acids, nucleic acids complexed with polypeptide or other molecules and nucleic acids immobilized onto solid phase particles. The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art. Transcription of the DNA encoding the polypeptides of the present invention by higher eukaryotes is increased by inserting an enhancer sequence into the vector. Enhancers are cis-acting elements of DNA, usually about from 10 to 300 by that act on a promoter to increase its transcription. Examples including the SV40 enhancer on the late side of the replication origin by 100 to 270, a cytomegalovirus early promoter enhancer, the polyoma enhancer on the late side of the replication origin, and adenovirus enhancers.

mabCAR. The CAR architecture may be any suitable architecture, as known in the art. The antigen recognition domain is typically derived from an scFv, which as described above selectively binds to the non-endogenous antigenic moiety. In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD28, 4-1BB, and OX40 or the signaling components of cytokine receptors such as IL7 and IL15. In particular embodiments, co-stimulatory molecules are employed to enhance the activation, proliferation, and activity of Treg cells produced by the CAR after antigen engagement. In specific embodiments, the co-stimulatory molecules are CD28, OX40, and 4-1BB and cytokine and the cytokine receptors are IL7 and IL15. The CAR may be first generation, second generation, or third generation CAR, in which signaling is provided by CD3ζ together with co-stimulation provided by CD28 and a tumor necrosis factor receptor (TNFr), such as 4-1BB or OX40), for example.

Spacer. A spacer region links the antigen binding domain to the transmembrane domain. It should be flexible enough to allow the antigen binding domain to orient in different directions to facilitate antigen recognition. The simplest form is the hinge region from an immunoglobulin, e.g. the hinge from any one of IgG1, IgG2a, IgG2b, IgG3, IgG4, particularly the human protein sequences. Alternatives include the CH₂CH₃ region of immunoglobulin and portions of CD3. For many scFv based constructs, an IgG hinge is effective.

The length of the DNA linker used to link the scFv and zeta chain is important for proper folding. It has been estimated that the peptide linker must span 3.5 nm (35 Å) between the carboxy terminus of the variable domain and the amino terminus of the other domain without affecting the ability of the domains to fold and form an intact antigen-binding site. Many such linkers are known in the art, for example flexible linkers comprising stretches of Gly and Ser residues. The linkers used in the present invention include, without limitation, a rigid linker. In some specific embodiments of the invention, a rigid linker has the sequence SEQ ID NO:1 (EAAAK)n, where n is 1, 2, 3, 4, 5, 6, etc. In some specific embodiments, n is 3.

T2A peptide. T2A peptide can be used to link the CAR of the invention to an epitope tag or other protein or peptide, including without limitation a sortable tag. T2A-linked multicistronic vectors can be used to express multiple proteins from a single open reading frame. The small T2A peptide sequences, when cloned between genes, allow for efficient, stoichiometric production of discrete protein products within a single vector through a novel “cleavage” event within the T2A peptide sequence. Various 2A peptide sequences are known and used in the art, for example see Szymczak-Workman et al. (2012) Cold Spring Harb Protoc. 2012(2):199-204, herein specifically incorporated by reference. They are small (18-22 amino acids) and have divergent amino-terminal sequences, which minimizes the chance for homologous recombination and allows for multiple, different 2A peptide sequences to be used within a single vector.

Immune Responsiveness Modulators.

Immune checkpoint proteins are immune inhibitory molecules that act to decrease immune responsiveness toward a target cell. Expression of certain checkpoint proteins is frequently associated with Treg cells, e.g. CTLA4, GITR, LAG3, etc. The expression of these and other immune suppressive proteins may be upregulated in the engineered Treg cells of the invention, including upregulation by introduction of coding sequences for the proteins.

Cytotoxic T-lymphocyte-associated antigen 4 (CTLA4; also known as CD152) and programmed cell death protein 1 (PD1; also known as CD279)—are both inhibitory receptors. CTLA4 is expressed exclusively on T cells where it primarily regulates the amplitude of the early stages of T cell activation. CTLA4 counteracts the activity of the T cell co-stimulatory receptor, CD28. CD28 and CTLA4 share identical ligands: CD80 (also known as B7.1) and CD86 (also known as B7.2). The major physiological roles of CTLA4 are downmodulation of helper T cell activity and enhancement of regulatory T (TReg) cell immunosuppressive activity.

Other immune-checkpoint proteins are PD1 and PDL1. The major role of PD1 is to limit the activity of T cells in peripheral tissues at the time of an inflammatory response to infection and to limit autoimmunity. PD1 expression is induced when T cells become activated. When engaged by one of its ligands, PD1 inhibits kinases that are involved in T cell activation. PD1 is highly expressed on T_{Reg} cells, where it may enhance their proliferation in the presence of ligand.

Lymphocyte activation gene 3 (LAG3; also known as CD223), 2B4 (also known as CD244), B and T lymphocyte attenuator (BTLA; also known as CD272), T cell membrane protein 3 (TIM3; also known as HAVcr2), adenosine A2a receptor (A2aR) and the family of killer inhibitory receptors have each been associated with the inhibition of lymphocyte activity and in some cases the induction of lymphocyte anergy.

LAG3 is a CD4 homolog that enhances the function of T_{Reg} cells. LAG3 also inhibits CD8⁺ effector T cell functions independently of its role on T_{Reg} cells. The only known ligand for LAG3 is MHC class II molecules, which are expressed on tumor-infiltrating macrophages and dendritic cells. LAG3 is one of various immune-checkpoint receptors that are coordinately upregulated on both T_{Reg} cells and anergic T cells.

BTLA is an inhibitory receptor on T cells that interacts with TNFRSF14. The system of interacting molecules is complex: CD160 (an immunoglobulin superfamily member) and LIGHT (also known as TNFSF14), mediate inhibitory and co-stimulatory activity, respectively. Signaling can be bidirectional, depending on the specific combination of interactions.

A2aR, the ligand of which is adenosine, inhibits T cell responses, in part by driving CD4⁺ T cells to express FOXP3 and hence to develop into T_{Reg} cells.

Pancreatic survival enhancing factors. The constructs of the invention express one or more factors that enhance the viability and/or function of pancreatic cells. Factors of interest include, without limitation, GLP1, STIM1, prolactin, placental lactogen, PDGF A, PDGF B, GIP, and VEGFA. In some embodiments the factor is GLP1.

As used herein, “transplant rejection” or “autoimmune disease”, including type I diabetes, GVHD, etc. is defined as a functional and structural deterioration of the cell, tissue or organ due to an immune response expressed by the individual, and independent of non-immunological causes of dysfunction. “Tolerance”, for example refers to the failure to respond to an antigen. “Peripheral tolerance” refers specifically to tolerance acquired by mature lymphocytes in the peripheral tissues.

“Protection” which can be partial or complete, refers to a state in which the effects of rejection are less than they would be if tolerance had not been induced or enhanced. The invention permits grafts and hosts to survive what would otherwise be damaging or lethal events.

By “effective amount” or “effective dose” is meant the amount of engineered T cells sufficient to produce a clinically beneficial result in the treatment of animals, preferably mammals, and more preferably humans.

“Inhibition” refers to partial or complete blockade or prevention of one or more activities directly or indirectly leading to damage or rejection of a graft, or injury to a host due to an autoimmune response.

### Engineered Treg Cells

Embodiments of the invention include cells that express a mabCAR of the invention. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, particularly a Treg cell. As discussed above, the engineered Treg cells can also comprise exogenous coding sequences for immunomodulatory proteins.

The cells can be autologous cells, syngeneic cells, allogeneic cells and even in some cases, xenogeneic cells. In many situations one may wish to be able to kill the engineered Treg cells. For this purpose one can provide for the expression of certain gene products in which one can kill the modified cells under controlled conditions, such as inducible suicide genes.

In particular cases the individual is provided with therapeutic Tregs engineered to comprise a mabCAR of the invention. The cells may be delivered at the same time or at different times as another type of therapy. The cells may be delivered in the same or separate formulations as another type of therapy. The cells may be provided to the individual in separate delivery routes as another type of therapy. The cells may be delivered by injection at a lesion site or intravenously or orally, for example. Routine delivery routes for such compositions are known in the art.

Expression vectors that encode the mabCAR of the invention can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors or lentiviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some embodiments AAV, retroviral or lentiviral vectors are used to deliver the CAR of the invention to a T cell.

Adeno associated virus (AAV) is an attractive vector system for use in the cells of the present invention as it has a high frequency of integration and it can infect nondividing cells, thus making it useful for delivery of genes into mammalian cells, for example, in tissue culture or in vivo. AAV has a broad host range for infectivity. Details concerning the generation and use of rAAV vectors are described in U.S. Pat. Nos. 5,139,941 and 4,797,368, each incorporated herein by reference.

The cells may be administered as desired. Depending upon the response desired, the manner of administration, the life of the cells, the number of cells present, various protocols may be employed. The number of administrations will depend upon the factors described above at least in part.

Retroviruses are useful as delivery vectors because of their ability to integrate their genes into the host genome, transferring a large amount of foreign genetic material, infecting a broad spectrum of species and cell types and of being packaged in special cell lines.

Lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2 and the Simian Immunodeficiency Virus: SIV. Recombinant lentiviral vectors are capable of infecting non-dividing cells and can be used for both in vivo and ex vivo gene transfer and expression of nucleic acid sequences. In some embodiments the lentiviral vector is a third generation vector (see, for example, Dull et al. (1998) J Virol. 72(11):8463-71). Such vectors are commercially available. 2nd generation lentiviral plasmids utilize the viral LTR promoter for gene expression, whereas 3rd-generation transfer vectors utilize a hybrid LTR promoter, see, for example Addgene for suitable vectors.

### Methods

The Tregs that have been modified with the construct(s) are then grown in culture under selective conditions and cells that are selected as having the construct may then be expanded and further analyzed, using, for example; the polymerase chain reaction for determining the presence of the construct in the host cells. Once the modified host cells have been identified, they may then be used as planned, e.g. expanded in culture or introduced into a host organism.

Depending upon the nature of the cells, the cells may be introduced into a host organism, e.g. a mammal, including humans, in a wide variety of ways. The cells may be introduced at the site of the inflammatory lesion.

Targeting antibodies are administered to a subject prior to, or concurrent with, or after administration of the mabCAR expressing T cells. The targeting antibodies bind to target cells in the subject, e.g. sites of autoimmune or GVHD lesions. The targeting antibodies may be formulated for administered to a subject using techniques known to the skilled artisan. Formulations of the tagged proteins may include pharmaceutically acceptable excipient(s). Excipients included in the formulations will have different purposes depending, for example, on the nature of the tag, the protein, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

A formulation of targeting antibodies may include one type of targeting antibody, or more than one, such as two, three, four, five, six or more types of targeting antibodies. The different types of targeting antibodies can vary based on the identity of the antigenic moiety, the identity of the antibody, or both.

The targeting antibodies may be administered to a subject using modes and techniques known to the skilled artisan. Exemplary modes include, but are not limited to, intravenous, intraperitoneal, and intratumoral injection. Other modes include, without limitation, intradermal, subcutaneous (s.c., s.q., sub-Q, Hypo), intramuscular (i.m.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect such administration.

Formulations comprising the targeting antibodies are administered to a subject in an amount which is effective for treating and/or prophylaxis of the specific indication or disease. In general, formulations comprising at least about 0.1 mg/kg to about 100 mg/kg body weight of the tagged proteins are administered to a subject in need of treatment. In most cases, the dosage is from about 1 mg/kg to about 10 mg/kg body weight of the tagged proteins daily, taking into account the routes of administration, symptoms, etc.

In accordance with the present invention, a therapeutic composition of an engineered Treg cell expressing a mabCAR is administered as a therapy to a subject. The dose of Treg calls may be 10⁴, 10⁵, 10⁶, 10⁷, 10⁸, 10⁹ or more/kg body weight. The cells may be administered in any suitable excipient that maintains the viability of the cells.

In some embodiments the subject has been diagnosed with T2D or IDDM, or pre-IDDM or pre-T2D. One of skill in the art can determine the patients who would potentially benefit from a therapeutic agent that would reduce or prevent the development of overt diabetes. One of skill in the art can determine the therapeutically effective amount of the composition to be administered to a subject based upon several considerations, such as local effects, pharmacodynamics, absorption, metabolism, method of delivery, age, weight, disease severity and response to the therapy.

In some embodiments the subject has been diagnosed with T1D or IDDM, or pre-IDDM or pre-T1D. One of skill in the art can determine the patients who would potentially benefit from a therapeutic agent that would reduce or prevent the development of overt diabetes. One of skill in the art can determine the therapeutically effective amount of the composition to be administered to a subject based upon several considerations, such as local effects, pharmacodynamics, absorption, metabolism, method of delivery, age, weight, disease severity and response to the therapy.

In an embodiment of the present invention, the engineered Treg composition is administered in an effective amount to decrease, reduce, inhibit or abrogate inflammation of the pancreas and toxicity related to standard therapy, in combination with an effective dose of targeting antibodies. The amount of antibody in the composition may vary from about 1 ng to about 1 g, more preferably, 0.1 mg to about 100 mg.

Treatment regimens may vary as well, and often depend on the health and age of the patient. Certain types of disease will require more aggressive treatment, while at the same time, certain patients cannot tolerate more taxing regimens. The clinician will be best suited to make such decisions based on the known efficacy and toxicity (if any) of the therapeutic formulations.

In specific embodiments, the composition is given in a single dose or multiple doses. The single dose may be administered daily, or multiple times a day, or multiple times a week, or monthly or multiple times a month. A series of doses may be administered daily, or multiple times a day, weekly, or multiple times a week, or monthly, or multiple times a month.

The improvement is any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of an immune response against the recipient.

An improvement in pancreatic inflammation, e.g. IDDM, is also any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of immune response from the donor's cells, tissue and/or organ against the host's tissues.

In order to increase the effectiveness of oral administration of the composition of the present invention, these compositions may be combined with conventional therapy.

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment. To prepare pharmaceutical compositions, an effective amount of the antibody may be dissolved or dispersed in a pharmaceutically acceptable carrier or aqueous medium. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils.

The proper fluidity can be maintained, for example, by the use of a coating, such as lecithin, by the maintenance of the required particle size in the case of dispersion and by the use of surfactants.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with various of the other ingredients enumerated above, as required, followed by filtered sterilization.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose.

These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The targeting antibodies may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered. In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means. Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

All references cited in this specification are hereby incorporated by reference in their entirety. The following examples are solely for the purpose of illustrating one embodiment of the invention.

The invention will further be illustrated in view of the following figures and example.

### EXPERIMENTAL

Immune-mediated diseases, such as autoimmune diseases, can be catastrophic for patients. Many autoimmune diseases like type 1 diabetes remain refractory to today's best-of-care immunosuppressive therapies.

Current immunosuppressive medications such as steroids, cyclosporine or blocking monoclonal antibodies have broad activity and are not localized to sites of inflammation. These agents all act by blocking immune cell activation signals. A fundamentally different approach is to use regulatory T cell therapy (Tregs) cell therapy to treat immune diseases. Tregs actively suppress immune responses and do not just block activation of immune cells. Tregs in pre-clinical models and a few clinical trials have effectively controlled dysregulated immune responses. However, major challenges to the clinical implementation of Tregs remain: (a) Tregs must be more effectively targeted to tissue sites of immune attack, (b) Tregs must be better insulated against inactivation by inflammatory cytokines, and (c) Tregs must be better controlled from being too active for too long.

Recent advances in the genetic modification of T cells have led to promising cellular therapies, including those with engineered chimeric antigen receptor (CAR) T cells. In CAR T cell therapy, DNA constructs are transduced into T cells usually using viral vectors. A CAR fusion protein is expressed that possesses a surface antibody-binding domain and an internal cell-signaling domain. T cells are thus “re-wired” to recognize and destroy defined surface antigens such as the B cell surface receptor CD19. CAR T cells have proven very effective in treating otherwise untreatable B cell leukemia and lymphoma.

Provided herein is a new therapeutic platform of CAR Tregs that are directed against the Fc portion of prior administered therapeutic monoclonal antibodies, allowing for precise control of Treg immune regulatory activity and which further express pro-survival factors for the targeted tissue.

There are different types of Tregs, including the well described CD25 FOXP3 natural subset (nTreg). nTregs are enhanced by transducing them to express engineered CAR that have a surface domain that recognizes fluorescein conjugated to the Fc “back-end” of mAbs. These CAR Tregs are active against mAbs that target specific tissue markers (e.g. CD31). The CAR also has an internal activation domain that activates Tregs even in inflamed tissues. The CAR nTregs will also express surface receptors that enhance suppressive capacity. nTreg activity is controlled by mAb dosing and once a cure is achieved no further mAb is needed.

In one example provided herein, mabCAR expresses a ScFV that recognizes FITC, fused to a CD28 costimulatory domain. Any monoclonal antibody (mAb) coupled to FITC within its Fc domain can be recognized. This approach was shown to be effective with T cells and Treg to ameliorate GvHD, and counter inflammation. mabCAR transfected conventional T cells (Tcon) expressed higher levels of CD44 and CD25 activation markers and produced more IFNγ after contact with FITC-mAb, demonstrating that mabCAR binding to the antibody drives cell activation.

MabCAR luc+ donor splenocytes directed against CD26 are injected into an IDDM animal model. To confirm mabCAR ability to divert homing in different models, we injected mabCAR murine B cells in a xenogeneic model where immune deficient NSG animals were engrafted with human pancreatic islets into the left kidney capsule. In conclusion, the mabCAR expression by immune cells can be used to control cell homing after adoptive transfer in different models according to localizing mAb availability. MabCAR approach provides a new tool for optimizing T cell and Treg based cellular therapies to modulate and control IDDM.

### Example 1

Engineer the md-CAR construct, transduce Tregs and show suppressive capacity using in vitro cell culture. md-CAR transduced nTregs will activate preferentially against cell culture plates coated with mAbs-FITC, or against cultured cells with bound mAbs-FITC.

md-CAR Construct.

All lentiviral constructs are based on the self-inactivating pHR vector. The sequences encoding 1×9Q (anti-FITC scFV) protein as well as LAG-3 and CTLA-4 are separated from one another by the nucleotide sequence of a foot-and-mouth disease virus derived 2A peptide which results in stoichiometric production of equimolar amounts of several proteins from a single mRNA.

Virus Production.

Lentiviral particles are produced as previously described and viral titers determined by infection of 293T cells with serial dilutions of the vector stock.

T_{reg} Sorting.

Following Miltenyi bead (MAC) enrichment of CD25⁺ T cells, we will then stain for CD4 (GK1.5, eBioscience) and sort to >97% purity on a FACSAria or FACSAria II flow cytometer (BD Biosciences).

Lentiviral Transduction.

Concentrated anti-FITC scFV as well as anti-FITC scFV-CTLA-4-LAG-3 containing lentivirus is added to the cells (MOI=10-20), in the presence of polybrene (2 μg/ml). The mixture of cells and lentivirus is plated in 96-well U-bottom plates (Corning) at 5×10 cells/100 μl/well and centrifuged at 1000×g for 2 hours at 25° C. and subsequently cultured at 37° C. for an additional 24-48 hours.

Cell Culture.

Cells are cultured in the presence of IL-2, mAb-FITC and target cells or anti-CD3/CD28 for 96 hours at 37° C.

Treg Cytokine Production.

At the end of the culture cells are stained according BED Pharmingen cytokine staining protocol with anti-TGF-beta1, IL-10 and IFN-gamma mAbs using isotype antibodies as controls and FACS analyzed. Supernatants are also harvested and analyzed using Milliplex Mouse Cytokine/Chemokine magnetic bead premixed panel (Millipore) through Luminex bio-plex 200 system for detecting and quantifying secreted cytokines.

CFSE Proliferation Assay of Treg.

Before setting up cultures, cells are incubated with CellTrace™ CFSE (Life Technologies) for 10 min at 37° C. and 5 min with cold medium on ice. At each cell division CFSE dye will be split by the dividing cells, as assessed through FACS analysis according CSFE dye signal intensity.

Statistical Analysis:

We will compare in vitro activity of md-Tregs in the following conditions: (1) unstimulated with target cells (2) unstimulated with target cells with bound mAb-FITC and (3) stimulated with anti-CD3/CD28 (positive control). All conditions will have 3-5 replicate wells, for at least 3 repeated experiments.

To confirm mabCAR ability to divert homing in different models, we injected mabCAR murine B cells in a xenogeneic model where immune deficient NSG animals were engrafted with human pancreatic islets into the left kidney capsule. mabCAR murine B cells were directed against the human CD26 molecule expressed by islet α-cells and were detectable specifically in the islet human graft 2 weeks after injection.

In conclusion, the mabCAR expression by immune cells can be used to control cell homing after adoptive transfer in different models according to localizing mAb availability. We believe that mabCAR approach may represent a new tool for optimizing T cell and Treg based cellular therapies to modulate and control GvHD.

### Example 2

Improved Allograft Tolerance Directed by T Cells Expressing Chimeric Antigen Receptor

Cellular therapies based on permanent genetic modification of T cells have emerged as a promising strategy for cancer. However it remains unknown if modification of T cells or T cell subsets like regulatory cells (Treg) could control responses to allogeneic targets. Here we use transient expression of a chimeric antigen receptor (CAR) that recognizes labeled monoclonal antibodies to control T cell activation in vivo. Expression of monoclonal antibody directed CAR (mAbCAR) permitted targeted activation of donor T cells at specific tissue sites, and mitigated graft-versus-host disease—without impacting anti-neoplastic T cell activity. mAbCAR Treg activated by MHC Class I on allografted islets promoted striking targeted protection and survival of transplanted islets, and subsequent MHC-specific prevention of skin graft rejection. Thus, transient genetic modification to produce mAbCAR T cells led to durable immune-modulation, suggesting therapeutic strategies for controlling alloreactivity in settings like organ transplantation.

Recent advances in the genetic modification of T cells have led to promising cellular therapies, including engineering T cells to express chimeric antigen receptor (CAR). T cells expressing surface CARs have been directed to selected specific antigens expressed by neoplastic cells, inducing an antigen-mediated immune response, including tumor regression and elimination. Using this approach, CAR T cells have been emerged as a new cell based therapy for several otherwise untreatable hematologic cancers, and have ushered in a new era for CAR-based cancer specific immune cell therapy.

More recently, CAR technology has been also studied aiming to modulate immune responses in non-neoplastic disease settings. CAR T cells with inhibitory function had the capacity to control excessive or “off target” responses by CAR T cells. Regulatory T cells, subpopulations of T cells with regulatory function, have been engineered to express CARs aiming to suppress antigen specific immune responses in different diseases. CD4⁺CD25⁺FoxP3⁺ regulatory T cells (Treg) are a subpopulation of T cells that can suppress conventional CD4⁺ and CD8⁺ T cells (Tcon) proliferation and function of multiple other immune cell types, and regulate physiological immune responses. The use of Treg adoptive transfer in pre-clinical models and in the clinic has been shown to effectively prevent graft versus host disease (GvHD), a life-threatening complication of hematopoietic stem cell transplantation (HCT) involving donor cell-mediated immune attack of host tissues. However, major challenges to the clinical implementation of Treg cell-based therapies remain. For example, (1) to limit off-target effects, Treg must be more effectively targeted to tissue sites of immune attack, (2) Treg require ex vivo or in vivo activation to enhance their function, and (3) functional Treg cells may require expansion for practical clinical applications since they are a relatively rare population in peripheral blood.

Here we used advances in T cell genetic engineering to permit expression of a CAR in Tcon and Treg that binds antibodies conjugated with fluorescein isothiocyanate (FITC) fluorochrome in the Fc region (hereafter ‘mAbCAR’). This permitted efficient modular use of previously-characterized monoclonal antibodies (mAbs) conjugated with FITC that (1) bound mAbCAR, (2) activated T cell function in vitro and in vivo, and (3) promoted homing of T cells expressing mAbCAR to specific antigens and cells. We used the mAbCAR approach to divert Tcon and Treg homing after adoptive transfer in transplantation models in order to modulate and control GvHD while maintaining graft versus tumor (GvT) effects. In mouse models mAbCAR expressing Treg cells directed to MHC-mismatched pancreatic islets prolonged allograft survival and elicited alloantigen-specific tolerance to secondary skin grafts. This work highlights the flexibility and the immune modulatory properties of mAbCAR Treg cells to control alloreactivity and suggests these cells are a new tool for potential translation purposes in T cell based immunotherapies and tolerance induction.

A Flexible mAb-Directed CAR to Generate T Cells and Treg.

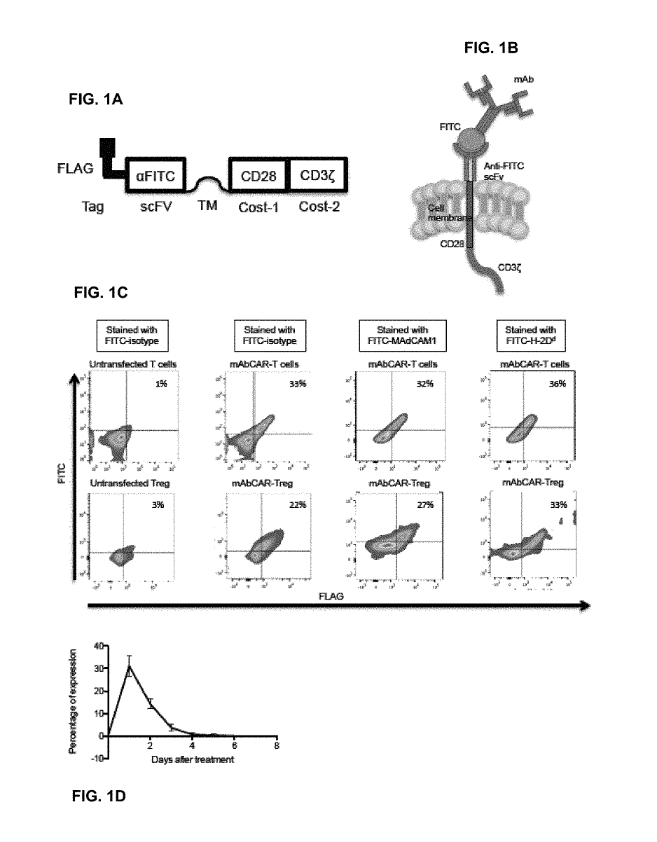

To exploit the exquisite specificity of mAbs, we built a chimeric antigen receptor that could be activated by binding to mAbs. To do this we generated a CAR (clone 1×9Q) that specifically binds FITC, a standard mAb molecular conjugate. Briefly, we fused an anti-FITC scFv portion to murine CD28 and CD3, costimulatory domains and named this synthetic receptor ‘mAbCAR’ (FIG. 1A-B). To facilitate detection of mAbCAR expression we also incorporated the FLAG immunoepitope in mAbCAR (FIG. 1A). CD28 is a costimulatory molecule widely tested in CAR T cell function with established roles in suppression by Treg.

We used transient transfection to express the genomic DNA plasmid transgene encoding mAbCAR in primary T cells and Treg. Transfection efficiency and surface expression kinetics were assessed by flow cytometric analysis by incubating FITC-conjugated isotype control mAb and an anti-FLAG mAb and quantifying FLAG₊ and FITC₊ cells. Both Tcon and Treg expressed mAbCAR at efficiencies of ˜30% (FIG. 1C). The level of FITC-conjugated antibody binding did not detectably depend on the specific antibody used (FIG. 1C). As expected, mAbCAR expression in T cells and Treg peaked then was almost completely lost after approximately 3-4 days (FIG. 1D).

mAbCAR T Cell Binding of FITC Induces Activation.

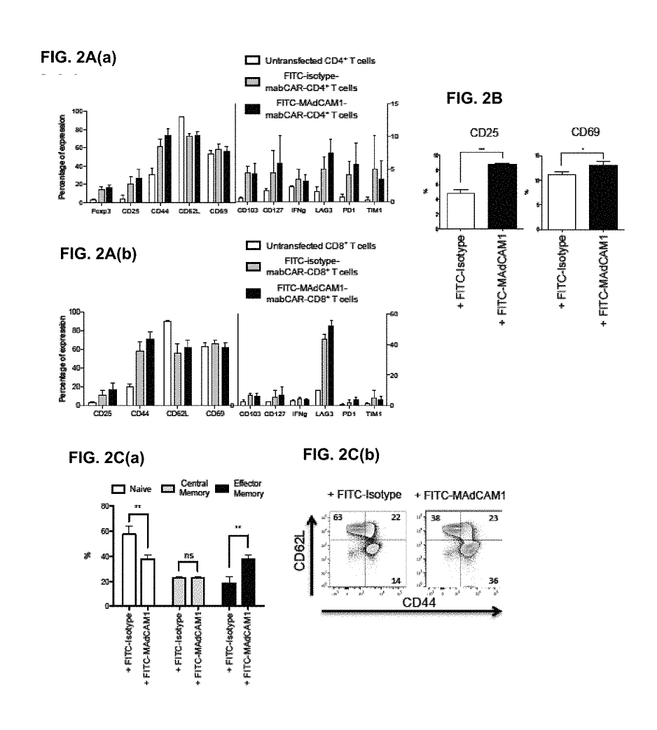

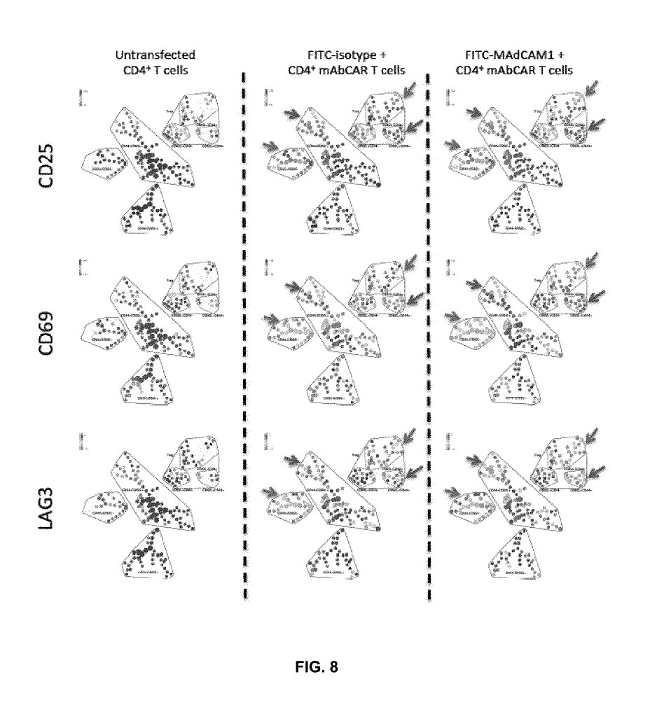

To evaluate the activation of mAbCAR T cells by FITC binding, we purified mAbCAR T cells by flow cytometry and incubated these in vitro with different FITC-conjugated antibodies (KLH/G2a-1-1 or MECA-367) attached to a tissue culture well. Protein and phenotypic markers of T cell activation were quantified by mass cytometry. In the presence of FITC conjugated antibodies, both CD4⁺ and CD8⁺ mAbCAR-T cells showed statistically significant increased activation with upregulation of CD44, CD25 and Lag3 and the production of cytokines such as IFNγ (FIG. 2A). Using SPADE analysis, we found that FITC mediated activation was more pronounced in the effector memory subpopulations of both the observed CD4⁺FoxP3^{neg} and CD4⁺FoxP3⁺ mAbCAR T cells (FIG. 8). Thus, mAbCAR T cells were specifically activated in response to FITC.

We next evaluated the specificity of mAbCAR T cells activation using FITC-conjugated monoclonal antibody against MAdCAM1, a known cell-surface integrin mainly expressed in the endothelium of the gut and secondary lymphoid tissues such as lymph nodes and spleen. MAdCAM1 is also expressed by a small subset of primary splenocytes and we used these as target cells for assaying MAdCAM1-mAbCAR T cells. We compared purified mAbCAR T cells incubated with FITC-conjugated MECA-367, a mAb specific for MAdCAM1, to T cells incubated with FITC-conjugated isotype control (KLH/G2a-1-1) mAb. After coculture of mAbCAR T cells with syngeneic C57Bl/6 (H-2b) mouse splenocytes, we found that MAdCAM1-mAbCAR T cell activation, quantified by expression of CD25 and CD69, was greater than activation of isotype-mAbCAR T cells (FIG. 2B). Moreover, a higher percentage of MAdCAM1-mAbCAR T cells acquired an effector memory phenotype (CD44⁺CD62L^{neg}, FIG. 2C). These findings suggest that mAbCAR T cell activation can be directed by mAbs that specifically bind cell surface antigen.

mAbCAR T Cell Targeting Directs T Cell Localization and Modulates GvHD.

T cell homing dynamics after adoptive transfer are critical for the development of GvHD and tissue-specific T cells have been shown to play a crucial role in GvHD pathophysiology. Thus, we tested whether transient expression of our mAbCAR could durably influence T cell reconstitution after HCT and mitigate GvHD severity. We hypothesized that targeting different cell surface antigens would produce distinct patterns of tissue localization by activated mAbCAR T cells, and we used in vivo bioluminescent imaging (BLI) after adoptive transfer to track luciferase-expressing (luc⁺) mAbCAR T cells. MAdCAM1 is expressed by the gut endothelium, and has important roles in GvHD development and lethality. By contrast, SDF1 (also known as CXCL12), is expressed in the bone marrow, spleen and liver but not in the gut. Moreover, we did not detect expression of either MAdCAM1 or SDF1 on our mAbCAR T cells by flow cytometry. Thus, we evaluated mAbCAR T cells pre-incubated with anti-MAdCAM1 antibody or with anti-SDF1 antibody.

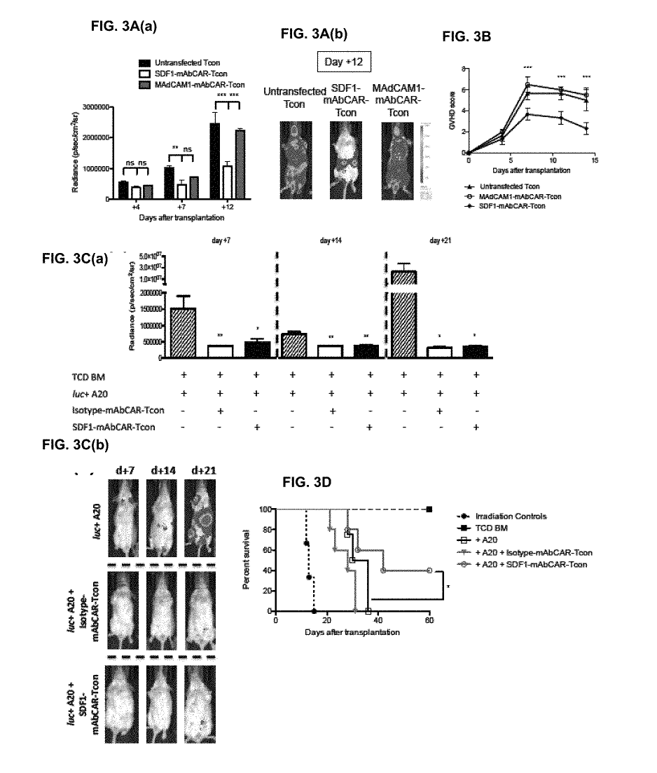

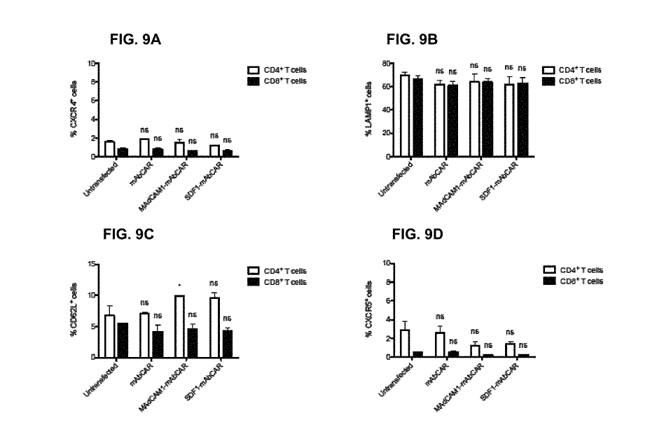

We adoptively transferred 1.0×10⁶ cells MAdCAM1-mAbCAR or SDF1-mAbCAR T cells and T cell depleted bone marrow (TCD BM) from C57Bl/6 (H-2^{b}) donors into lethally irradiated allogeneic BALB/c (H-2^{d}) recipients. All the mice reached complete donor engraftment. Compared to untransfected control T cells, in vivo imaging showed that MAdCAM1-mAbCAR T cells had similar gut bioluminescence intensity. In contrast, SDF1-mAbCAR T cells showed a distinct pattern of localization to other organs, including liver, spleen and bone marrow (FIG. 3A). SDF1-mAbCAR T cell recipient mice had significantly improved GvHD scores and weight profiles (FIG. 3B) compared to mice that received MAdCAM1-mAbCAR T cells or control mice. Moreover, while there were lasting differences in clinical outcome between SDF-1 and MAdCAM1 directed T cells, FACS analysis of mAbCAR T cells reisolated after transfer did not reveal detectable differences in production of surface homing molecules like CXCR4, CXCR5, CD62L or LPAM1 (FIG. 9). Together our findings suggest that specific antibodies and mAbCAR T cells can be used to direct T cell tissue localization after adoptive transfer to modulate GvHD outcomes.

Eliciting GvT effects is one important therapeutic goal of adoptive T cell transfer, but it was unclear whether GvT activity was affected in mAbCAR T cells. To assess this, SDF1-mAbCAR T cells or isotype-mAbCAR T cells were transferred into transplanted allogeneic BALB/c mice that also received an intravenous infusion of luc₊ A20 leukemia cells. Recipient mice that received only A20 cells died because of tumor progression, mice that received A20 cells and isotype-mAbCAR T cells cleared the tumor, but quickly died because of GvHD. Mice that received A20 cells and SDF1-mAbCAR T cells cleared the tumor and had a better overall survival (FIG. 3C-D). Thus, mAbCAR T cells coupled to FITC-anti-SDF1 antibody had reduced GvHD lethality while retaining GvT activity.

mAbCAR Treg Cells Retain Suppressive Activity.

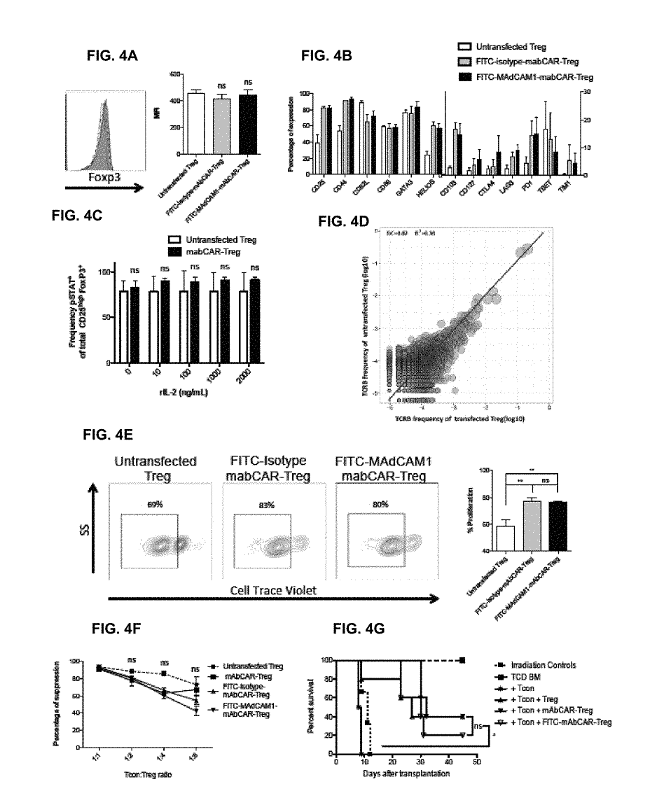

We next tested if mAbCAR expression and mAb targeting could modulate natural CD4⁺CD25⁺FoxP3⁺ Treg activation and in vivo behavior, to assess use of mAbCAR for Treg based tolerance induction. To test if mAbCAR expression altered the phenotype and immunoregulatory function of Treg, we evaluated both freshly isolated Treg and Treg expanded by in vitro culture (see Methods). Transient transfection and activation of mAbCAR Treg cells with different FITC conjugated antibodies in vitro did not significantly change FoxP3 expression (FIG. 4A), but did induce an increased expression of several activation markers correlated with suppressive function including CD25, CTLA4, and LAG3 (FIG. 4B).

mAbCAR Treg showed similar levels of phosphorylated STAT5 compared to control untransfected Treg (FIG. 4C). Likewise we did not detect significant differences in TCR repertoires of the mAbCAR-transduced versus sham-transduced Treg populations (FIG. 4D). However activation of mAbCAR Treg with FITC-conjugated antibodies in vitro led to significantly enhanced T cell receptor-dependent proliferative responses following T cell activation to anti-CD3/CD28 beads (FIG. 4E). This suggests that mAbCAR stimulation of Treg does not induce anergy but rather augments TCR-mediated expansion. Since polyclonal Treg can activate in response to distinct allogeneic antigens and potentially become alloantigen-specific, mAbCAR stimulation could potentially facilitate the onset and persistence of peripheral tolerance. Together, our findings suggest Treg expressing mAbCAR retain hallmark Treg phenotypes.

To test if the enhanced capacity to activate and proliferate conferred by mAbCAR might alter the suppressive capacity of Treg, we assayed Treg suppression of Tcon proliferation. As expected, unmodified cultured Treg suppressed the proliferation of Tcon (activated by anti-CD3/CD28 beads) in a dose-dependent manner (FIG. 4F). Similarly we found that cultured mAbCAR Treg also suppressed proliferating Tcon in a dose-dependent manner. Thus, mAbCAR expression and FITC-binding does not interfere with Treg suppressive function in vitro (FIG. 4F).

To assess the suppressive capacity of mAbCAR Treg in vivo, we adoptively transferred donor mAbCAR Treg (exposed to FITC-MAdCAM1 antibody or FITC-isotype-control antibody) from C57Bl/6 mice into lethally irradiated allogeneic BALB/c recipients prior to allogeneic donor Tcon and TCD BM. Both MAdCAM1- and isotype-mAbCAR Treg were able to effectively prevent GvHD and their efficacy was comparable to untransfected Treg in terms of prolonged mouse survival (FIG. 4G). Together our in vitro and in vivo findings confirm that cardinal Treg functions are maintained in Treg expressing mAbCAR and activated by FITC-conjugated antibodies.

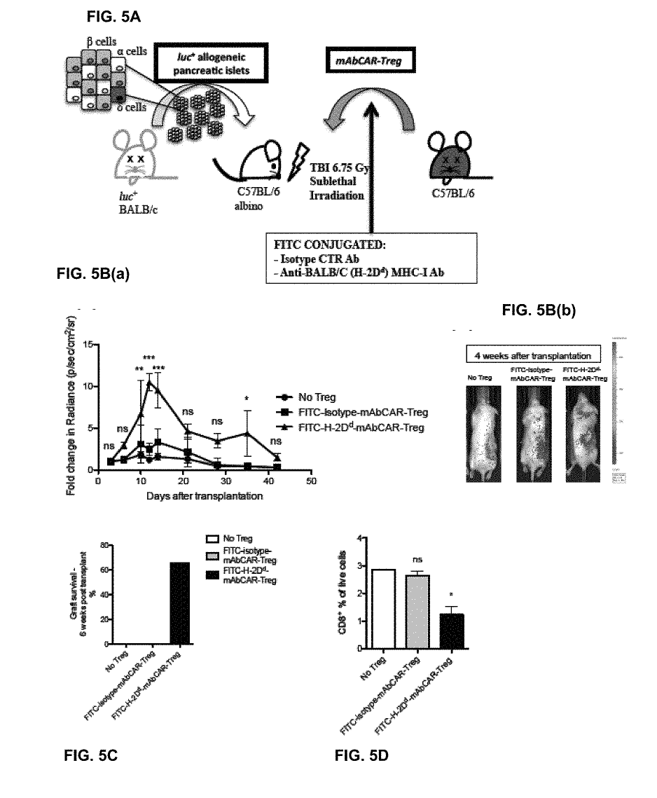

Allogeneic islet tolerance induced by mAbCAR Treg directed against islet alloantigen Based on our findings, we postulated that mAbCAR could be used to target Treg to transplanted allogeneic pancreatic islets and protect them from allograft rejection. We transplanted allogeneic luc+ pancreatic islets from BALB/c (H-2^{d}) donors in the right subcapsular renal space of sublethally irradiated C57Bl/6-albino (200 gray; H-2^{b}) mice (FIG. 5A). Donor islet survival was assessed by BLI and post-mortem histology. To direct Treg function to transplanted islets, we transferred recipient-derived (H-2b) mAbCAR Treg bound to mAbs directed against donor H-2D^{d} MHC-1 antigen expressed only by donor BALB/c derived islets (“H-2D^{d}-mAbCAR Treg”: FIG. 5A). Compared to mice receiving isotype mAbCAR Treg or no Treg after islet transplantation, mice that received H-2D^{d}-mAbCAR Treg showed significantly enhanced islet survival (p=0.002; FIG. 5B-C). FACS analysis of cell suspensions obtained from re-isolated renal islet grafts showed reduction in host type CD8 T cell infiltration in the presence of FITC-H-2D_{d}-mAbCAR Treg at day +10 after islet transplantation (FIG. 5D). These data provide strong evidence that mAbCAR Treg directed to allogeneic islet grafts can significantly improve allograft survival.

H-2D^{d}-mAbCAR Directed Treg Localize in Proximity to Allogeneic Pancreatic Islet Grafts.

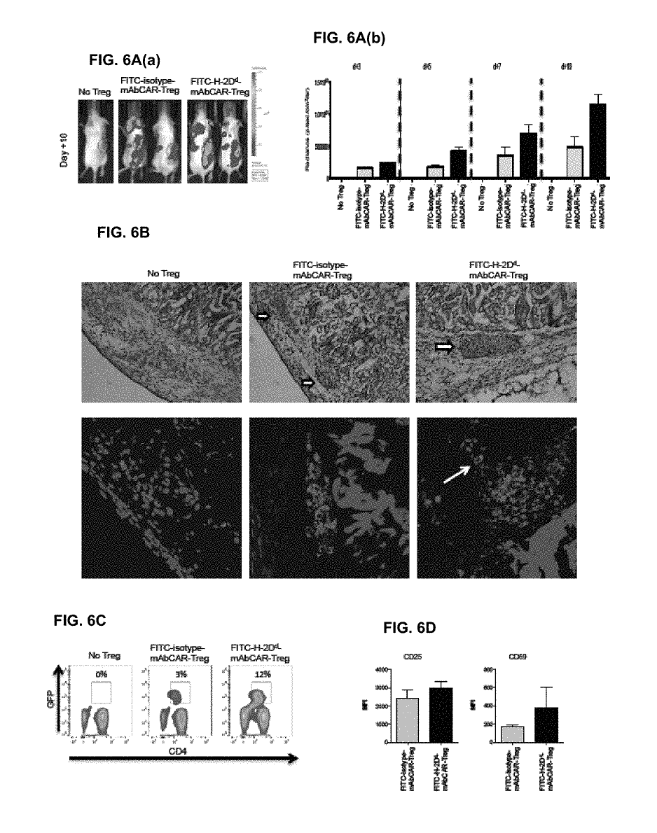

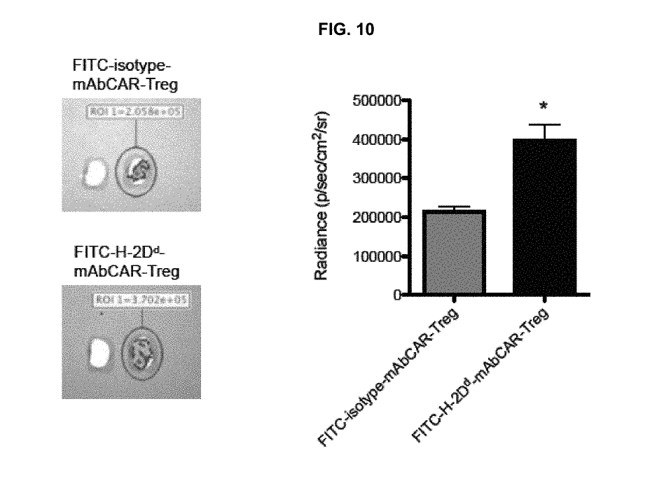

To demonstrate localization by H-2D^{d}-mAbCAR Treg to islet allografts, we used islets from donor wild-type BALB/c animals and mAbCAR Treg from recipient C57Bl/6 mice that express both luciferase and green fluorescent protein (GFP⁺). BLI revealed that the bioluminescent signal intensity of H-2D^{d}-mAbCAR Treg was significantly greater compared to isotype-control mAbCAR Treg (FIG. 6A). Ex vivo BLI of the isolated right kidney at day +10 after islet graft transplantation confirmed greater signal in kidneys from mice that received allogeneic islets and H-2D^{d}-mAbCAR Treg (FIG. 10).

Confocal microscopy analysis confirmed the presence of GFP⁺ H-2D^{d}-mAbCAR Treg adjacent to, or within transplanted islets at day +10 after adoptive transfer indicating an increased ability to home in selected target tissues (FIG. 6B). H-2D^{d}-mAbCAR Treg expansion was also detectable in secondary lymphoid organs of the same animals (FIG. 6C). In evaluation of splenocytes at day +10 after islet transplantation and adoptive transfer of H-2D^{d}-mAbCAR Treg, we found that Treg showed increased expression of markers such as CD69 and CD25 showing a highly activated status (FIG. 6D). However, as expected, FLAG immunoepitope expression was undetectable by immunostaining, confirming loss of expression of the original FLAG-tagged mAbCAR construct. These results demonstrate that H-2D^{d}-mAbCAR Treg persist within the targeted islet graft tissue. Unexpectedly and importantly, this increased specificity of localization to allografted islets persisted long after 72 hours, when transduced Treg lose the surface expression of the mAbCAR construct (see FIG. 1D). Thus, despite transient mAbCAR expression, we observed durable mAbCAR Treg localization, and evidence of suppressive Treg function.

mAbCAR Treg Acquire Antigen Specificity after Adoptive Transfer In Vivo.

Based on observations here of (1) durable peripheral tolerance to allogeneic islets after mAbCAR Treg transfer and (2) proliferation of mAbCAR Treg upon TCR stimulation, we hypothesized that H-2D^{d}-mAbCAR Treg might foster antigen-specific peripheral tolerance of tissue allografts by Treg. To test this, we evaluated survival of secondary skin grafts, a rigorous assay of peripheral tolerance in this setting. Specifically, we grafted skin on C57Bl/6− albino mice 30 days after they had received allogeneic BALB/c islets and recipient-derived H-2D^{d}-mAbCAR Treg or BALB/c islets and isotype-control mAbCAR Treg. Each albino C57Bl/6 recipient mouse then received two skin grafts, one from an allogeneic BALB/c mice (that was H-2D^{d}, MHC-matched with the previous islets) or allogeneic FVB/n mice (not MHC-matched with the previous islets and therefore “third-party”). Mice that previously received isotype mAbCAR

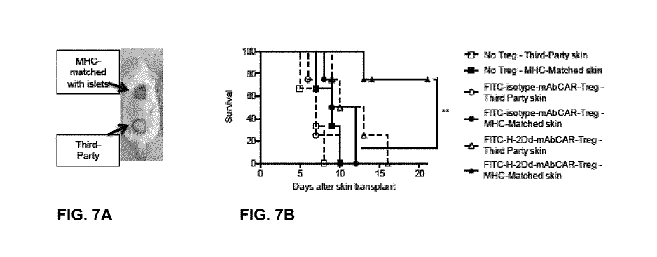

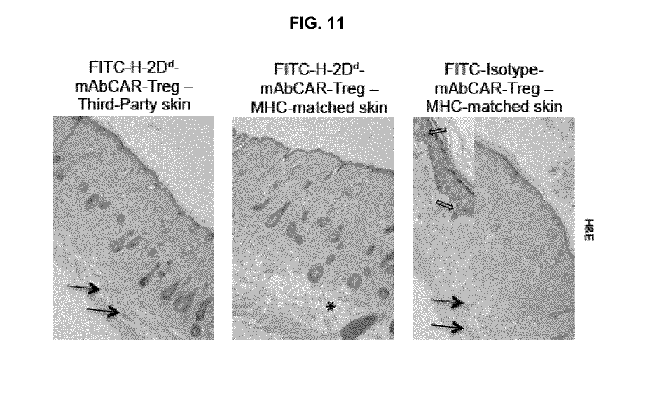

Treg showed quick rejection of both the skin grafts, but mice that were infused with H-2D^{d}-mAbCAR Treg showed a statistically significant prolonged survival of only the MHC matched BALB/c derived skin grafts, measured in a blinded fashion (FIG. 7A-B). Histology of the skin grafts at 2 weeks after skin transplantation showed the MHC-matched BALB/c grafts that lacked detectable signs of rejection compared to the “third-party” grafts (FIG. 11). These data further demonstrate that H-2D^{d}-mAbCAR Treg acquired durable antigen specificity suppressive function even though mAbCAR expression was transient. Together our data demonstrate that mAbCAR and similar approaches can be used to promote, target and enhance Treg function and peripheral immune tolerance induction, with striking improvements in tissue or organ allograft survival.

The genetic modification of T cells to direct their activation to specific targets in vivo has become a successful strategy for cancer immunotherapy, but relatively less is known about how adoptive transfer of activated CAR T cells or Treg might influence the recipient immune system in clinical settings like GvHD or allotransplant tolerance. To address these questions, we developed a system for expressing a CAR (mAbCAR) that binds to FITC and activates Treg functions, enabling flexible, modular targeting. Based on reliable, covalent coupling of FITC to mAbs, we tested and identified multiple antibodies that permitted both targeting and activation of T cells and Treg. A similar approach was recently reported to potentiate anti-cancer effects of T cells (see Ma et al. (2016) PNAS 113(4) E450-8; Tamada et al. (2012) Clin Canc. Res. 18(23) 6436-45).

Here we used multiple systems to demonstrate that the introduction and transient expression of a CAR construct that into a fraction of adoptively-transferred T cells or Treg permitted remarkable experimental control of T cell localization and activity to improve outcomes of experimental disease models in mice, including GvHD, GvT effect, and tissue allograft tolerance. For example, expression of the mAbCAR construct that directed T cell activation modulated the severity of GVHD. Mice that received CAR T cells directed to activate at the site of the gut (MAdCAM1) showed lethal gut GvHD, while those that received CAR T cells directed to activate mainly within the bone marrow compartment (SDF1) showed less GVHD, without impairing GvT responses or bone marrow engraftment. It is striking that incomplete, transient transfection of donor T cells could have a lasting effect on T cell reconstitution and GvHD, providing new avenues for T cell graft engineering in HCT paradigms. Previous CAR T cell strategies have involved expression of an external targeting ScFV antibody binding domain fused with internal signaling domains of the costimulatory factors CD28 and CD3ζ. There is some evidence that CD28 costimulation of Treg can modulate their activation and recent studies suggest that CD28/CD3ζ internal stimulatory domains can enhance Treg function.

Most studies of CAR T cell antigen specificity involve CAR-directed T cell activation against tumor-antigen-expressing cell targets in pre-clinical models. Comparatively little is known about CAR-T cell mediated approaches to modulate immune responses and modify disease microenvironment. Nothing is yet known about how CAR Treg might alter immune responses or their interplay with antigen-specific tolerance. Here we investigated the hypothesis that CAR Treg cells directed by the exquisite specificity of mAbs could achieve target antigen-specific and target cell-specific immune-modulatory effects.

First we proved that mAbCAR Treg maintain their suppressive phenotype and function (see FIG. 4), thus transfected Treg were able to suppress T cell proliferation both in vitro and in vivo. In this context MAdCAM1- and isotype-mAbCAR Treg were as effective as untransfected Treg in reducing GvHD lethality in mice that received allogeneic T cell transfer. It is relevant to clarify that mAbCAR Treg were only a fraction (˜30%) of the total injected Treg.

To evaluate the use of CAR Treg in islet transplantation, we investigated the effect of transient expression of CAR in Treg directed to MHC-mismatch in islet allografts. In clinical settings like leukemia, the relentless growth of target cells justifies use of stably transduced CAR T cells. However, we postulated that stable transduction using viral vectors or other strategies may not be practical for immunomodulation in settings like allograft tolerization. For example, polyclonal Treg cell subsets could become persistently activated and too immunosuppressive. Surprisingly, we found that Treg localization and allograft tolerization persisted long after the transient expression of the CAR construct. Even more remarkably, the one-time introduction of transiently expressing CAR Treg resulted in antigen-specific peripheral tolerance, as MHC-matched secondary skin grafts were accepted while “third party” grafts were rejected. These results are particularly relevant as they were observed in mice that received a skin graft 3 weeks after the previous islet transplant when Treg expansion had already occurred and when Treg had completely lost the mAbCAR expression. Therefore mAbCAR-mediated transient binding induces antigen specificity that persists even if the initiating mAbCAR function itself is lost. Our findings suggest that transient transfection of Treg might be useful for immunosuppression while avoiding complications of Treg overactivation. Since polyclonal Treg may activate in response to specific allogeneic antigens and potentially become alloantigen-specific, mAbCAR stimulation may facilitate the formation of peripheral tolerance by “super-charging” alloreactive Treg.

We also observed that mAbCAR Treg targeting luc₊ allogeneic pancreatic islets showed an unexpected and significant increase in BLI signal from the grafts that started in the very first days after transplant. Multiple cellular mechanisms could account for an improvement in this signal, including the possibility that the presence of a Treg induced cytokine environment could promote both engraftment and islet β-cell proliferation. This opens the exciting possibility that CAR Treg could provide multiple signals to improve islet transplantation outcomes.

In preclinical GvHD models adoptive transfer of polyclonal CD25⁺ Treg has been associated with improved antigen-specific tolerance but these models depend upon impractically-high doses of Treg that have yet to be achieved in human translational science. Moreover, evidence from clinical trials that improved antigen-specific tolerance may result from Treg transfer is lacking. Immunotherapy with polyclonal Treg has multiple challenges including (1) lack of mechanisms to ‘target’ cells to sites of inflammation, (2) inactivation of suppressive functions and possible conversion to pro-inflammatory cells at these sites, and (3) short-lived antigen-specific immune protection. Our studies provide unprecedented evidence that genetically-modified CAR Treg could be used to overcome each of these problems. Our findings also raise the possibility that additional regulatory immune cell populations, including invariant natural killer T cells Tr1 cells and others, could be used in mAbCAR-based approaches.

Antibody tagging systems (e.g. nanocapsules, DNA bridges, Fc-tagging sequences) that proved to be safe in different clinical applications could be adapted for overcoming such limitation. The use of stable transduction and ‘kill-switches’ or the use of other transient transfection strategies could also be evaluated.

In summary, we describe a flexible, modular system for modifying CAR T cells that allows targeted homing to specific antigens and cells and activation of in vivo functions. In an animal model, this system was useful for targeting inflammation, and achieving durable antigen-specific immune protection of tissues, including allografted pancreatic islets. Future uses of mAbCAR Treg strategies in human could address persistent urgent problems in clinical medicine needing targeted immunomodulation and preservation of tissue survival and function.

### Materials and Methods

Mice.

We performed experiments using gender-matched mice between 8 and 16 weeks old. BALB/c (H-2^{d}), C57BL/6 (H-2^{b}), and FVB/N (H-2^{q}) mice were purchased from Jackson Laboratories (Sacramento, Calif.). luc₊ BALB/c mice were generated as described and rose in the Stanford Animal Facility. C57BL/6 albino FoxP3 mutant mice expressing luciferase and green fluorescent protein (FoxP3^{luc/GFP}) were a kind gift from Dr. Günter J. Hammerling (Heidelberg, Germany) and bred in our animal facility. All animal experiments were performed in accordance with guidelines from the Stanford University Institutional Animal Care and Use Committee.

Cell Isolation.

For CD4⁺ and/or CD8⁺ T cells we obtained cell suspensions from splenocytes and peripheral lymph node cells of donor animals and we enriched them with anti-CD4 and/or anti-CD8 magnetic-activated cell sorting (MACS; Miltenyi Biotec, Auburn, Calif.) For TCD BM we flushed long bones by injecting PBS and we after depleted T cells with anti-CD4 and anti-CD8 MACS beads. For Treg we obtained pooled cell suspensions from spleens and lymph nodes, stained for CD25-allophycocyanin (APC) and CD4, enriched with anti-APC MACS beads and sorted for CD4⁺CD25^{bright} cells or for CD4⁺CD25⁺GFP⁺ cells from C57BL/6 albino FoxP3^{luc/GFP} mice on a FACS Aria or FACS Aida (BD Biosciences, San Jose, Calif.). Purity of the final Treg product was always >95% CD4₊FoxP3₊ cells when using both the approaches.

Flow Cytometric Analysis and Mass Cytometry Analysis.

For flow cytometry we purchased from Southern Biotech (Birmingham, Ala.), BDbioscences (San Jose, Calif.), eBioscience (San Diego, Calif.), and Biolegend (San Diego, Calif.) the following antibodies: CD4 (GK1.5), CD8 (53-6.7), CD25 (PC61), FoxP3 (FJK-16s), H-2D^{d} (34-2-12), MAdCAM1 (MECA-367), SDF1 (2B11/CXCR4), IgG1 (KLH/G2a-1-1), IgG2a (G155-178). We used anti-mouse/rat FoxP3 Staining Set (eBioscience) for intranuclear Foxp3 staining and Fixable Viability Dye eFluor® 506 (eBioscence) for dead cell staining. Analysis was performed on a LSR II (Becton-Dickinson, San Jose, Calif.). For mass cytometry we used the antibody list as previously reported [48]. For intranuclear factors we used anti-mouse/rat FoxP3 Staining Set (eBioscience) and for staining dead cells we use Cisplatin (Sigma). Analysis was performed on Cytof and data were analyzed through cytobank as previously described.

In Vitro Cell Culture.

Treg or Tcon have been plated in 96-well or 48-well flat-bottom plates containing cRPMI, interleukin-2 (IL2, 50 IU/ml for Tcon; 1000 IU/ml for Treg) and anti-CD3/CD28 beads (Dynabeads, Invitrogen, 1:10 bead:cell ratio for Tcon; 1:2 bead:cell ratio for Treg). Tcon have been cultured for 2-4 days, washed and then transfected. Treg have been cultured for 18 consecutive days, checked every 6 days for purity by FACS analysis (CD4⁺FoxP3⁺ cells constantly >90%), washed, and then transfected.

Transfection of T Cells and Treg and Incubation with FITC-Conjugated Antibody.

T cells and Treg were transfected using MIRUS transfection reagents as per manufacturer's protocol (Mirus Bio LLC, Madison, Wis.). Briefly, cells were plated at a concentration of 5×10⁵ cells/ml and were incubated with the 1×9Q DNA as well as the MIRUS reagent for 24-48 hrs to allow for expression of the chimeric receptor. Transfected cells were then washed and incubated or not in vitro with the FITC conjugated antibody of interest for 30 minutes on ice. FITC conjugated antibodies that have been used for stimulation of mAbCAR T cells and/or mAbCAR Treg are the followings: H-2D^{d} (34-2-12), MAdCAM1 (MECA-367), SDF1 (2B11/CXCR4), IgG1 (KLH/G2a-1-1), IgG2a (G155-178). Cells were then washed once more and injected into mice (0.5-1×10⁶ cells/mouse).

Bone Marrow Transplantation, GvHD and Tumor Models.

For mouse model of GvHD, BALB/c recipient mice were irradiated with total body irradiation (TBI) 2 doses of 4 Gy, 4 hours apart with 200-Kv X-ray source, and rescued with 5×10⁶ TCD BM cells from allogeneic C57Bl/6 mice. GvHD was induced with 1×10⁶ Tcon from C57Bl/6 mice injected at day 0. Transplanted animals were kept in autoclaved cages with antibiotic water or antibiotic food (sulfamethoxazole-trimethropim; Schein Pharmaceutical, Corona, Calif.). C57Bl/6 Treg were injected at different time points and at different doses as reported. For BLI analysis of cell proliferation luc₊ Tcon or Treg have been used accordingly.

In the tumor model with A20 leukemia cell line, luc₊ 2×10₅/mouse A20 cells were injected together with TCD BM after lethal irradiation. Mice received different mAbCAR Tcon populations as reported in the text and tumor growth was assessed in vivo by BLI. In vivo BLI was performed as described with an IVIS 29 charge-coupled device imaging system (Xenogen, Alameda, Calif.). Images were analyzed with Living Image Software 4.3.1 (Xenogen). Mouse survival was reported and mice were weighed weekly and GvHD score was calculated (Measurement of phosphorylation of signal transducer and activator of transcription 5 (pSTAT5). FACS-purified, in vitro expanded and transfected Treg were cultured overnight in complete RPMI without IL-2 supplementation. The cells were then washed in PBS (ThermoFisher, Waltham, Mass.). pSTAT5 was detected as described previously. Briefly, the cells were pulsed with 0, 100, 1000 and 2000 IU/mL of recombinant interleukin-2 (rIL-2, Teceleukin, Hoffmann-La Roche, Nutley, N.J.) for 15 min before fixation with 4% paraformaldehyde (Sigma-Aldrich, St. Lois, Mo.). Perm Buffer III from BD Biosciences was used for permeabilization. The following antibodies were used: Alexa Fluor 647-conjugated anti-STAT5 (pY694, BD biosciences, San Jose, Calif.), Brilliant Violet 650-conjugated anti-CD4 (RM4-5, Biolegend, San Diego, Calif.), allophycocyanin-cyanine 7-conjugated anti-CD25 (PC61), anti-FoxP3 (FSK-16s), phycoerythrin-conjugated TAG (L5). Data was collected on a FACSAria (BD) and the data was analyzed using FlowJo Vx software (Treestar, Ashland, Oreg.).

TCR Repertoire Sequencing and Data Processing.

Total RNA was isolated from both transfected and untransfected Treg with Qiagen RNeasy Micro kit. Rapid amplification of 5′ complementary DNA ends(5′RACE) was employed to capture VDJ genes of TCRB. Briefly, first-strand cDNA was generated by Superscript II reverse transcriptase with oligo-dT30 and a universal oligo was added to the 5′end of mRNAs. cDNA was then amplified with a TCRβ primer from constant region and the 5′end universal primer. The library was constructed with KAPA Hyper Prep Kits (Kapa Biosystems). The sequencing was carried out with a 500-cycle MiSeq Reagent Kit v2 on a illumina Miseq machine. After removing primer sequences, we used MiXCR for VDJ rearrangement analysis and determine complementarity-determining region 3 (CDR3). CDR3 amino acid sequences and frequency were summarized from MiXCR outputs.

Mouse Islet Transplantation.

For islet transplantation experiments, 100 mouse islets from donors aged 2-4 months were transplanted per recipient mouse. Islets were resuspended in cold Matrigel and transferred into the renal capsular space of host animals using a glass microcapillary tube. Transplant recipients were 2- to 4-month-old male mice and were anesthetized using ketamine/xylazine. Appropriate depth of anesthesia was confirmed by lack of toe-pinch response. After 2 weeks, kidneys with grafts were removed, fixed in 4% paraformalde and processed for cryosectioning and immunohistology.

Immunostaining of Islet Transplant Grafts.

Primary antibodies used were guinea pig polyclonal anti-insulin (1:200, Dako, A0564) and secondary antibody used was donkey anti guinea pig (1:500, Jackson Immunoresearch, 706-605-148). Samples were imaged using an SP2 confocal microscope with a ×40 objective.

Antigen Specificity Response after In Vivo FITC-H-2D_{d}-mAbCAR-Treg Transfer.

To analyze the antigen specific response, mice received a secondary double skin graft MHC-matched with the previously transplanted pancreatic graft (BALB/c) and “third-party” (FVB/N). All animals were anesthetized with Ketamine/Xilasine at 21 days after pancreatic islet transplantation. Under sterile conditions, MHC-matched and “third-party” skins (1 cm×1 cm) were transplanted in dorsal site and sutured (4-0 Safil® Violet, B/Braum, Germany). Buprenorphine (0.05 mg/Kg) was administered subcutaneously as analgesia before and after skin transplantation every 24 h. Double skin graft survival was monitored in mice that received pancreatic islet graft and no Treg treatment (skin MHC-matched with islet graft, skin “third-party”), mice that received pancreatic islet graft and FITC-isotype-mAbCAR Treg (skin MHC-matched with islet graft, skin “third-party”), and mice that received pancreatic islet graft and FITC-H-2D_{d}-mAbCAR-Treg (skin MHC-matched with islet graft, skin “third-party”). Signs of onset of rejection, such as dryness, loss of hair, contraction, scaling, and necrosis were recorded for a period of time of 21 days after transplantation. Grafts were considered rejected when necrosis and detachment of the graft was observed. At this point the animals were euthanized, histological analysis of skin grafts by Hematoxilin-Eosin and CD4 staining were performed.

Statistical Analysis.

Log-rank test was used to detect differences in animal survival (Kaplan-Meier survival curves), while weight variation and GvHD score were analyzed with 2-way ANOVA test. All other comparisons were performed with the 2-tailed Student t test. p<0.05 was considered statistically significant.

What is claimed is:

1. A method for treating an inflammatory condition in a subject in need thereof, comprising administering to said subject:
an effective dose of regulatory T cells (Treg), are engineered to express a chimeric antigen receptor (CAR), that specifically binds to a non-endogenous antigenic moiety and encodes one or more pancreatic survival factors; and
an effective dose of targeting antibodies, which antibodies (i) bind to an antigen localized at a site of inflammation and (ii) are labeled with the antigenic moiety,
wherein inflammation is decreased at the targeted site.

2. The method of claim 1, wherein the antigenic moiety is a small molecule.

3. The method of claim 1 wherein the antigenic moiety is fluorescein isothiocyanate (FITC).

4. The method of claim 1, wherein the Treg cells are isolated from a peripheral blood sample.

5. The method of claim 4, wherein the Treg cells are expanded in culture.

6. The method of claim 1, wherein the CAR construct encodes one or more costimulatory molecules.

7. The method of claim 6, wherein the costimulatory molecule comprises the CD28 activating domain.

8. The method of claim 1, wherein the Treg is further engineered to comprise an exogenous construct that encodes or more T cell downregulatory proteins.

9. The method of claim 8, wherein the T cell downregulatory proteins comprise one or both of CTLA4 and LAG3.

10. The method of claim 1, wherein the inflammatory disease is insulin dependent diabetes mellitus (IDDM).

11. The method of claim 1 wherein the targeting antibodies specifically bind to CD26.

12. The method of claim 1 wherein the targeting antibodies specifically bind to MHC Class I molecules.

13. The method of claim 1 wherein the targeting antibodies specifically bind to a pancreatic islet autoantigen.

14. The method of claim 1 wherein the targeting antibodies specifically bind to a secreted factors selected from insulin, glucagon, SDF-1 and MCP-1.

15. The method of claim 1 wherein the targeting antibodies specifically bind to HIC-2B4.

16. The method of claim 10, wherein the engineered Treg cells express one or more of GLP1, STIM1, prolactin, placental lactogen, PDGF A, PDGF B, GIP, and VEGFA.

17. A cellular composition of engineered Treg cells for use in the method of claim 1.

18. A kit comprising the cellular composition of claim 17, and a suitable targeting antibody.
